(19) United States
(12) Patent Application Publication
(10) Pub. No.: US 2019/0119332 A1
(43) Pub. Date: Apr. 25, 2019

(54) METHODS TO PURIFY AVIDIN-LIKE PROTEINS AND FUSION PROTEINS THEREOF

(71) Applicant: THE CHILDREN'S MEDICAL CENTER CORPORATION, Boston, MA (US)

(72) Inventors: Fan ZHANG, Chestnut Hill, MA (US); Richard MALLEY, Beverly, MA (US); Yingjie LU, Chestnut Hill, MA (US)

(73) Assignee: THE CHILDREN'S MEDICAL CENTER CORPORATION, Boston, MA (US)

(21) Appl. No.: 16/098,258

(22) PCT Filed: May 4, 2017
(86) PCT No.: PCT/US2017/030969

§ 371 Date: Nov. 1, 2018

Related U.S. Application Data
(60) Provisional application No. 62/331,575, filed on May 4, 2016.

(51) Int. Cl. C07K 14/195; C07K 1/22; G01N 33/543; G01N 33/566

(52) U.S. Cl. CPC C07K 14/195; C07K 1/22; G01N 2333/195; G01N 33/566; G01N 33/543

(57) ABSTRACT

The present invention generally relates to separation matrix comprising a lipoic acid (LA) compound or derivative thereof for use in a method for purifying and isolating a biotin-binding protein, including fusion proteins and complexes thereof. Embodiments described herein relate to methods for reversible binding of a biotin-binding protein, e.g., rhizavidin, including fusion proteins and complexes thereof to a matrix comprising a lipoic acid (LA) compound or derivative thereof immobilized to a solid support, where the biotin-binding protein can be detached from the matrix, making it possible to isolate the biotin-binding protein efficiently and quickly and under a mild conditions while minimizing protein denaturation and maximizing protein purification and isolation.

### CROSS REFERENCED TO RELATED APPLICATIONS

This Application claims the benefit under 35 U.S.C. § 119(e) of U.S. Provisional Application Ser. No. 62/331,575 filed on filed May 4, 2016, the content of which is incorporated herein in its entirety by reference.

### FIELD OF THE INVENTION

The present invention generally relates to the field of affinity chromatography, and more specifically to separation matrix comprising a lipoic acid (LA) compound or derivative for use in a method for purifying and isolating a biotin-binding protein, including fusion proteins and complexes thereof. The disclosure also relates to methods for the separation of a biotin-binding protein, e.g., rhizavidin, including fusion proteins and complexes thereof with aforementioned matrix, with the advantage of efficiently using lipoic acid or a lipoic acid derivative immobilized to a support, thereby making it possible to isolate a target material efficiently under a mild condition in a short period.

### BACKGROUND OF THE INVENTION

Affinity chromatography allows for the purification of a protein of interest from a mixture of molecules, such as a cellular harvest, based on the preferential binding of the protein of interest to a target in solid phase, such as a gel matrix. This solid phase component typically is formed into a column through which the mixture containing the protein of interest is applied. In this initial step, called the capture step, the protein of interest specifically binds to the target in solid phase whereas other components in the mixture flow through the column.

There is a continuous need in medical practice, research and diagnostic procedures for rapid, accurate, isolation or quantitative determination of different isolation of avidin and avidin-like proteins from various biological fluids. The basic streptavidin-biotin interaction technique is utilized in affinity chromatography, cytochemistry, histochemistry, pathological probing, immunoassays, in-situ hybridization, bio-affinity sensors and cross-linking agents, as well as in more specific techniques such as targeting, drug delivery, flow cytometry and cytological probing.

Additionally, affinity chromatography is typically used as the first stage of a multi-stage purification process for a biotin-binding proteins, such as avidin or streptavidin, but not all affinity chromatography matrices for purifying these biotin-binding proteins are efficient or suitable for all biotin-binding proteins, due to either weak, or to tight, binding of the biotin-binding protein to the ligand on the affinity chromatography column or other reasons. Streptavidin or avidin, or modified proteins thereof, can readily be immobilized on surfaces to capture, separate or detect biotinylated moieties, e.g. biotin-labeled, or biotin-derivative-labeled proteins or cells from crude, complex mixtures (see e.g., US Application 2008/0255004, U.S. Pat. Nos. 5,395,856, 5,691,152). Similarly, biotin or biotin derivatives or biotin-analogues can also readily be immobilized on surfaces to isolate, capture biotin-binding proteins, such as streptavidin or avidin proteins or complexes or fusion proteins containing the same biotin-binding proteins (see., e.g. US Application 2008/0255004).

However, biotin-streptavidin (or avidin) linkage results in an essentially irreversible binding of the two binding partners, which is not suitable for affinity chromatography purification of such biotin-binding proteins. This the high affinity necessitates the use of harsh chemical reagents and complex procedures, e.g. boiling in high salt conditions or use of formamide and EDTA heated to 94° C. for several minutes (Tong & Smith, Anal. Chem. 64: 2672-2677, 1992), or 6 molar guanidine HCl, pH 1.5 to achieve partial or complete bond disruption. The use of such conditions to reverse the biotin-streptavidin linkage is therefore generally undesirable, especially in the purification of proteins or separation of cells, bacteria and viruses etc. when it is important to preserving the cells integrity and maintain viability or infectivity, or for affinity chromatography where such conditions increase the denaturation of the purified protein. Additionally, affinity chromatography purification using the biotin-streptavidin (or avidin) linkage typically use streptavidin or avidin-like protein attached to a solid support in methods to isolate biotin-tagged proteins and biomolecules.

Much of the focus in the field has been directed to strategies to disrupt or reverse the biotin-streptavidin linkage (Lee & Vacquier, Anal Biochem. 206: 206-207, 1992, Elgar & Schofield, DNA Sequence 2: 219-226, 1992, and Conrad & Krupp, Nucleic Acids Res. 20: 6423-6424, 1992), there has been little with respect to methods to increase the efficiency and purification of biotin-binding proteins using affinity chromatography purification methods.

Methods to reduce the affinity of biotin to streptavidin or avidin includes generation of recombinant or chemically modified streptavidin or avidin. WO 01/05977, which describes mutant proteins produce a stable dimer. These stable dimers exhibit reversible biotin-binding properties when tested with 0.5 mM biotin in buffer (0.5% BSA; 0.5% Tween 20 and 1 M NaCl in PBS) at 37° C. for 1 hr.

U.S. Pat. No. 6,022,951 also describes a mutated recombinant streptavidin with reduced affinity for biotin, however, in order to disrupt the streptavidin-biotin bond of the mutated streptavidin, between 0.1 mM to 10 mM of biotin is needed. In addition, elution must be performed at either a high or a low pH, in high salt, or in the presence of ionic detergents, dissociating agents, chaotropic agents, organic solvents, protease (protease K) for at least 1 hour, resulting in increased risk of protein denaturinataion of the isolated protein.

U.S. Pat. Nos. 6,391,571; 6,312,916; and 6,417,331 describe muteins of avidin and streptavidin having a reduced binding affinity for biotin, but when these muteins are attached to a Spherosil-NH2 column, an elution buffer 50 mM ammonium acetate at a pH 3.0 or/and a gradient of 9 to 10 mM iminobiotin or biotin or an elution buffer comprising PBS buffer, pH 7.2 and a gradient of 0 to 10 mM biotin is required to elute the biotinylated compound.

There have been other reports of peptides with binding activity for streptavidin. U.S. Pat. No. 5,506,121 describes the generation of such peptides (Strep-tags) which can be eluted from a streptavidin agarose columns using a solution of 1 mM iminobiotin or 5 mM lipoic acid. U.S. Pat. No. 6,103,493 describes streptavidin muteins which can be attached to an affinity chromatography column, where the streptavidin-binding peptides be competitively eluted by other streptavidin ligands e.g. biotin, iminobiotin, lipoic acid, desthiobiotin, diaminobiotin, HABA (hydroxyazobenzene-benzoic acid) or/and dimethyl-HABA in a step-wise manner by applying 10 ml each of diaminobiotin, desthio-biotin and biotin at a concentration of 2.5 mM.

Accordingly, while there are many methods to efficiently isolate biotin-binding proteins such as streptavidin and avidin, either when they are present alone, or when they exist as part of a biotin-containing or biotin-derivative complex, such methods are not efficient or effective at isolating other non-avidin or non-streptavidin biotin-binding proteins. While His-tags and other protein purification tags can be used to isolate proteins, these are not suitable for GMP Compliant Purification of Proteins, or clinical grade protein purification. For GMP compliant protein production and purification, typical purification methods involve size exclusion, precipitation (e.g., using aluminum sulfate and the like) and require time consuming optimization to be tailored to the specific protein to be purified. Therefore, such methods are neither efficient or readily adaptable or suitable for GMP purification of a range of fusion proteins.

Accordingly, there is a need in the art for a method and systems for readily isolating other biotin-binding proteins, including for GMP Compliant protein purification. Despite advances made to date, there still exists a need for new and improved methods for selectively isolating and releasing other biotin-binding proteins. None of the previously reported methods for reversible binding between biotin and streptavidin or avidin are optimal or efficient for use in reversible binding between biotin and different biotin-binding proteins (i.e., biotin-binding proteins that are not streptavidin or avidin). Consequently, there is a continuing need in the art for alternative methods for reversibly and reliably isolating other biotin-binding proteins (i.e., biotin-binding proteins that are not streptavidin or avidin) and a modified affinity chromatography method to concentrate a range of biotin-binding proteins which allows for the use of proportionally smaller, less costly columns and fewer subsequent purification steps.

### SUMMARY OF THE INVENTION

The disclosure herein generally provides methods, compositions and kits for efficient and robust affinity chromatography (also referred to herein as affinity separation) of a biotin-binding protein, such as a biotin-binding domain, as well as a matrix for such affinity chromatography and washing methods.

Affinity chromatography is often used as the first stage of a multi-stage purification process for a biotin-binding domain, such as a rhizavidin protein or fusion protein or complex thereof, and the purity of the biotin-binding domain, such as a rhizavidin protein or fusion protein or complex thereof after affinity chromatography notably influences the kind and number of subsequent purification steps. Another important role for affinity chromatography is to concentrate the product, which allows for the use of proportionally smaller, less costly columns in subsequent purification steps. Therefore, it is particularly important to optimize the removal of impurities during the affinity chromatography step.

As disclosed herein, the inventors assessed the binding of rhizavidin to several ligands (e.g., biotin and biotin-derivatives) for use in affinity chromatography purification. While rhizavidin is known to bind to biotin and other biotin-related or biotin-derivatives, the inventors surprisingly discovered that the biotin-derivatives HABA (hydrooxyazobenzene-benzoic acid) or dimethyl-HABA, which bind with high affinity to streptavidin, did not bind to rhizavidin. Therefore, while rhizavidin and strepatavidin are similar, the inventors surprisingly discovered that ligands that bind to streptavidin do not necessarily bind to rhizavidin. The inventors also demonstrated that affinity columns comprising HABA or dimethyl-HABA (i.e., biotin derivatives that bind to strepatavidin) were not effective at purifying rhizavidin. Moreover, the inventors surprising discovered that only lipoic acid was effective for efficient purification of a rhizavidin protein or rhizavidin-containing fusion protein.

Accordingly, in one aspect, the disclosure herein provides a method of producing a purified biotin-binding protein of interest, e.g., a biotin-binding domain using an affinity chromatography (AC) matrix comprising lipoic acid (LA) compound to which a biotin-binding protein of interest is bound, the method comprising (i) contact a matrix comprising a lipoic acid (LA) compound (referred to herein as a “LA-matrix”) with a solution comprising the biotin-binding protein of interest, (ii) washing the LA-matrix to remove the non-bound proteins, and (iii) eluting the biotin-binding protein bound to the LA-matrix with one or more wash solutions as disclosed herein. In some embodiments, the biotin-binding protein of interest is loaded onto the LA-matrix prior to washing with the one or more wash solutions and the protein of interest is eluted from the LA-matrix after washing with the one or more wash solutions, in particular, to remove impurities from the LA-matrix.

Accordingly, the disclosure herein relates to methods, kits and compositions comprising a lipoic acid (LA) compound immobilized on the surface of a solid support to isolate a biotin-binding protein, such as, for example, a rhizavidin protein or a protein comprising a rhizavidin protein, e.g., a fusion protein or complex comprising a rhizavidin protein. In some embodiments, the lipoic acid (LA) compound is attached to second moiety, e.g., an antibody or bead, and in some embodiments, the antibody or bead can be attached to a solid support. Accordingly, the methods, compositions and kits as disclosed herein enable the separation and isolation of a biotin-binding protein, e.g., a biotin-binding domain, such as, but not limited to, a rhizavidin protein or a protein comprising a rhizavidin protein, such as, e.g., a fusion protein or complex comprising a rhizavidin protein from the rest of the components in the mixture.

Accordingly, one aspect of the disclosure herein relates to reversibly immobilizing a rhizavidin protein or a fusion protein comprising a rhizavidin protein to a solid support, comprising contacting the support with the rhizavidin protein or a fusion protein comprising a rhizavidin protein, wherein the solid support comprises a lipoic acid (LA) compound on the surface of the solid support. In some embodiments, the rhizavidin protein or a fusion protein thereof is contacted with, or binds to the solid support in a solution having a pH between 5.5-9.0, or a solution comprising 1M NaCl. In some embodiments, the method further comprises contacting the solid support comprising the immobilized rhizavidin protein or rhizavidin protein fusion protein with an elution buffer comprising 1-10 mg/ml of a lipoic acid (LA) compound to release the rhizavidin protein or fusion protein thereof from the solid support.

Another aspect of the disclosure herein relates to a method for purifying a rhizavidin protein or a fusion protein comprising a rhizavidin protein to a solid support, comprising: (i) contacting a solid support that comprises a lipoic acid (LA) compound on the surface of the solid the support with solution comprising a rhizavidin protein or a fusion protein comprising a rhizavidin protein; (ii) incubating for a sufficient amount of time to allow the a rhizavidin protein or a fusion protein comprising a rhizavidin protein to bind to the lipoic acid (LA) compound; (iii) washing the solid support comprising the lipoic acid (LA) compound on the surface of the solid the support with a wash solution to remove non-bound rhizavidin protein or a fusion protein comprising a rhizavidin protein; (iv) contacting the solid support comprising the immobilized rhizavidin protein or rhizavidin protein fusion protein with an elution buffer comprising 1-10 mg/ml of a lipoic acid (LA) and separating portions of the elution buffer which comprises the rhizavidin protein or fusion protein released from the solid support from portions of the elution buffer which do not comprise the rhizavidin protein or fusion protein released from the solid support; and (iv) collecting the portion of the elution buffer which comprises the rhizavidin protein or fusion protein thereof.

In some embodiments, the method for reversibly immobilizing a rhizavidin protein or a fusion protein thereof to a solid support, or a method for purifying a rhizavidin protein or a fusion protein thereof further comprises eluting the rhizavidin protein or rhizavidin protein fusion protein from the solid support by contacting the solid support comprising the immobilized rhizavidin protein or rhizavidin protein fusion protein with an elution buffer comprising 1-10 mg/ml of a lipoic acid (LA) compound to release the rhizavidin protein or fusion protein thereof from the solid support.

In all aspects described herein, a solution comprising a rhizavidin protein or a fusion protein thereof which contacts the solid support comprising the lipoic acid (LA) compound has a pH between 5.5-9.0, or a solution comprising 1M NaCl. In all aspects described herein, an elution buffer for eluting the rhizavidin protein or fusion protein thereof comprises between 1.0-10 mg/ml of a lipoic acid (LA) compound to release the rhizavidin protein or fusion protein thereof from the solid support.

Another aspect of the present invention relates to a composition, for example comprising a solid support, a lipoic acid compound and a Rhizavidin protein or Rhizavidin fusion protein, wherein the lipoic acid compound is attached to the solid support, and the Rhizavidin protein or Rhizavidin fusion protein comprises at least amino acids of SEQ ID NO: 1 or protein of at least 80% sequence identity to SEQ ID NO: 1 and is bound to the lipoic acid compound.

In some embodiments, the composition further comprises a buffer solution having a pH between 5.5-9.0, and/or 1M NaCl. In some embodiments, the composition is configured as an affinity chromatography column, for example, where a container in the shape of a column, having an upper inlet and a lower outlet comprises the solid support, the lipoic acid compound and the Rhizavidin protein or Rhizavidin fusion protein.

Another aspect of the present invention relates to an affinity chromatography column comprising the composition comprising a solid support, a lipoic acid compound and a Rhizavidin protein or Rhizavidin fusion protein wherein the lipoic acid compound is attached to the solid support, and the Rhizavidin protein or Rhizavidin fusion protein comprises at least amino acids of SEQ ID NO: 1 or protein of at least 80% sequence identity to SEQ ID NO: 1 and is bound to the lipoic acid compound.

Another aspect of the present invention relates to an affinity chromatography column comprising a LA-resin, wherein the LA-resin comprises a solid support and a lipoic acid compound attached to the solid support.

Another aspect of the present invention relates to a method of method of making a lipoic acid resin (LA-resin), comprising (a) contacting the solid support with a solution comprising a lipoic acid compound that has been activated for crosslinking to the solid support and incubating for a sufficient amount of time to allow the lipoic acid compound to cross-link to the solid support; and (b) removing the solution added in step (a), or transfer the solid support and cross-linked lipoic acid compound to a new purification column. In some embodiments, the solution comprising a lipoic acid compound that has been activated for crosslinking and is used in step (a) comprises Sulfo-NHS (N-hydroxysulfosuccinimide) and EDC, and can optionally be at pH 7.0.

Another aspect of the present invention relates to an affinity chromatography column produced by any of the methods as disclosed herein, wherein the solid support and cross-linked lipoic acid compound are present in a purification column (e.g., an affinity chromatography column).

In all aspects described herein, a Rhizavidin protein for purification and/or isolation comprises amino acids of SEQ ID NO: 1 or protein of at least 80% sequence identity to SEQ ID NO: 1.

In some embodiments, the solid support is selected from the group consisting of: plastic, glass, ceramics, silicone, metal, cellulose, membranes and gels, and can be, for example, a particle or a magnetic particle, such as a SEPHAROSE™ bead or similar such beads, e.g., agarose. In some embodiments, the solid support is in the form of any of: particles, sheets, dip-sticks, gels, filters, membranes, microfibre strips, biochips, tubes, wells, plates, fiber or capillaries, comb, pipette tip, microarrays. In further embodiments, the composition of any of claims 27 to 35, wherein the solid support is a polymeric material selected from the group of: agarose, SEPHAROSE™, cellulose, nitrocellulose, alginate, Teflon, latex, acrylamide, nylon membranes, plastic, polystyrene, glass or silica or metals.

In some embodiments, a lipoic acid compound is directly bound or linked to the solid support via a covalent bond, or alternatively, it can be indirectly linked to the solid support via a protein linker, peptide, nucleic acid, oligosaccharide, glycoprotein, or cross-linking reagent.

In some embodiments, the lipoic acid compound is lipoic acid or alpha-lipoic acid (ALA), and can be racemic lipoic acid, or enantiomerically pure or enantiomerically enriched R (+)-alpha-lipoic acid or S-(−)-alpha-lipoic acid. In some embodiments, the lipoic acid compound is a lipoic acid derivative, for example, but not limited to, a lipoic acid derivative is selected from the group of: Lipoylpyridoxamine, Lipoylpyridoxamine hydrochloride, Lipoylpyridoxamine hydrobromide, Lipoylpyridoxamine methanesulfonate, Lipoylpyridoxamine p-toluenesulfonate, 1,2-dithiolane analog, diethoxycarbonylated lipoic acid, 6,8-Bisacetylmercaptooctanoic Acid (Bis-acetyl Lipoic Acid), 6,8-Bisbenzoylmercaptooctanoic Acid (Bisbenzoyl Lipoic acid), 8-Acetylmercapto-6-mercaptooctanoic Acid (Monoacetyl Lipoate), 6,8-Biscarbamoylmethylmercaptooctanoic Acid, 6,8-Bis-[S—(N-methylsuccinimido)]mercaptooctanoic Acid.

Another aspect of the present invention relates to a kit comprising: (i) a lipoic acid compound attached to a solid support; and (ii) at least one reagent to remove an immobilized rhizavidin protein or fusion protein comprising a rhizavidin protein from the lipoic acid compound attached to the solid support. In some embodiments, the kit can further comprise an expression vector comprising the nucleic acid sequence for expression of a Rhizavidin fusion protein, wherein the nucleic acid sequence comprises (i) a nucleic acid sequence encoding a rhizavidin protein comprising SEQ ID NO: 1 or a protein of at least 80% sequence identity to SEQ ID NO: 1, and (ii) a nucleic acid comprising a multiple insertion site (MIS) for insertion of a nucleic acid sequence encoding a protein of interest to be fused to the Rhizavidin protein. In some embodiments, the nucleic acid can comprise a multiple insertion site (MIS) at the 5′- of the nucleic acid sequence encoding a rhizavidin protein comprising SEQ ID NO: 1 or a protein of at least 80% sequence identity to SEQ ID NO: 1, so that the protein of interest is at the N-terminus of the rhizavidin protein. In alternative embodiments, nucleic acid comprises a multiple insertion site (MIS) at the 3′- of the nucleic acid sequence encoding a rhizavidin protein comprising SEQ ID NO: 1 or a protein of at least 80% sequence identity to SEQ ID NO: 1, so that the protein of interest is at the C-terminus of the rhizavidin protein. In some embodiments, the expression vector further comprises a nucleic acid sequence comprising a lipidation sequence at the 5′ of the nucleic acid sequence encoding a rhizavidin protein comprising SEQ ID NO: 1 or a protein of at least 80% sequence identity to SEQ ID NO: 1. In some embodiments, the expression vector further comprises a nucleic acid sequence comprising a linker peptide between the nucleic acid sequence encoding a rhizavidin protein comprising SEQ ID NO: 1 or a protein of at least 80% sequence identity to SEQ ID NO: 1 and the nucleic acid comprising a multiple insertion site (MIS). In some embodiments, the protein of interest is an antigenic peptide or antigen polypeptide.

### BRIEF DESCRIPTION OF FIGURES

FIG. 1 shows the purification of His-tagged Rhizavidin protein comprising SEQ ID NO: 1 with either a Ni-NTA resin for purifying His-tagged proteins or a lipoic acid (LA) resin as described herein. Lane 9 shows the LA resin can be used to purify and isolate the His-tagged rhizavidin protein as efficiently and with a similar or greater yield to the His-tagged rhizavidin protein purified using the His binding to the Ni-NTA resin (in Lane 4).

FIG. 2 shows that Rhizavidin purified using the lipoic acid (LA) resin is a dimer in solution as determined by size exclusion column, the same as that purified from the Ni-NTA resin, where the Ni-NTA resin binds to the His tag on the Rhizavidin.

FIG. 3 shows that Rhizavidin purified using the lipoic acid (LA) resin can form a MAPS complex to biotinylated dextran. Lane 2 and 3 showed the position of Rhizavidin purified from Ni-NTA resin on SDS gel without or with boiling. Lane 4 showed Ni-NTA resin purified Rhizavidin still forms a complex with biotinylated dextran without boiling at 100 C where as Rhizavidin is released from the complex under boiling condition (Lane 5). Lane 6 and 7 showed that lipoic acid resin purified Rhizavidin runs similarly as Ni-NTA resin purified Rhizavidin on SDS gel. Lane 8 (not boiled) and 9 (boiled) showed that lipoic acid resin purified Rhizavidin forms complex with biotinylated dextran and the complex behaves the same as the one made with Ni-NTA resin purified Rhiavidin and biotinylated dextran.

FIG. 4 shows purification of a fusion protein of Rhizavidin-1500-0785 (i.e., a Rhizavidin protein comprising SEQ ID NO: 1 fused to a pneumococcal antigen selected from the pneumococcal proteins SP 1500 and/or SP 0785) lacking a His tag using the LA resin. The fusion protein is eluted in elutant samples 1-3 (lanes 5-7).

FIG. 5 shows that non-His tagged Rhizavidin-1500-0785 fusion protein purified using the lipoic acid (LA) resin is a dimer in solution as determined by size exclusion column, similar to the dimer that is purified using the Ni-NTA resin, where the Ni-NTA resin binds to the His tag on the Rhizavidin-1500-0785.

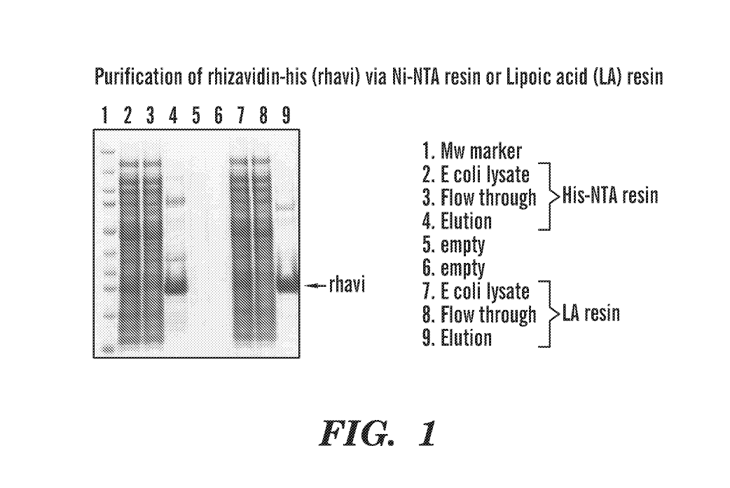

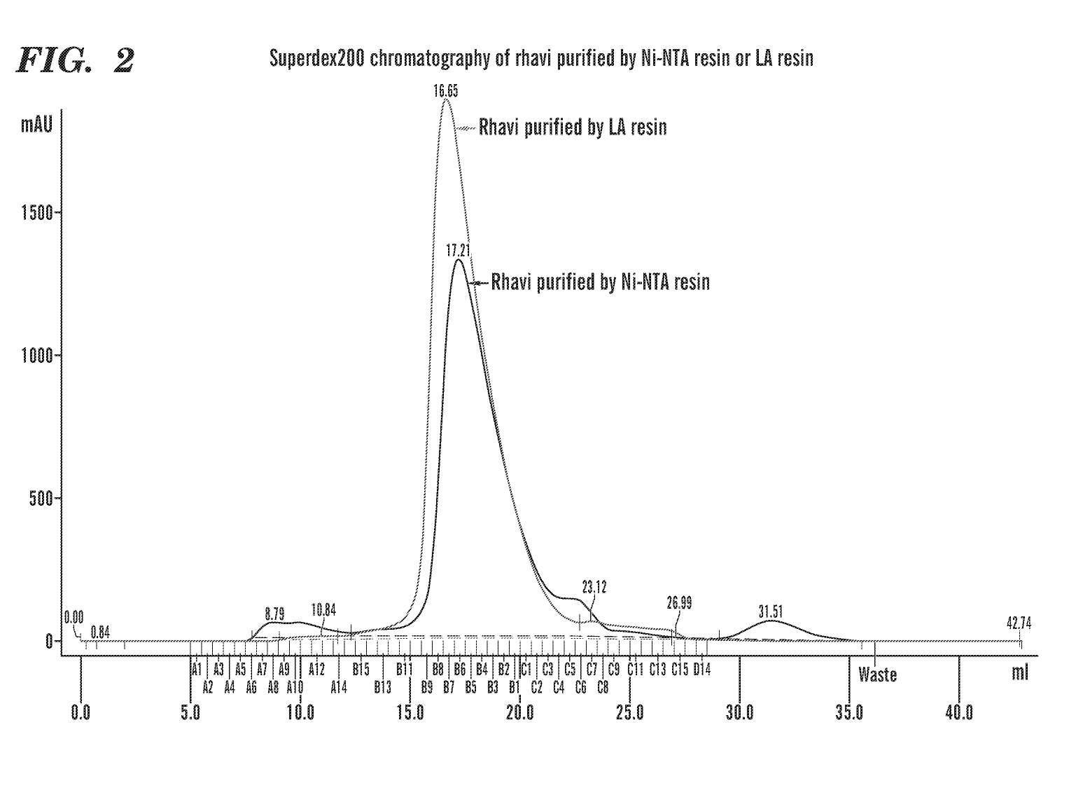

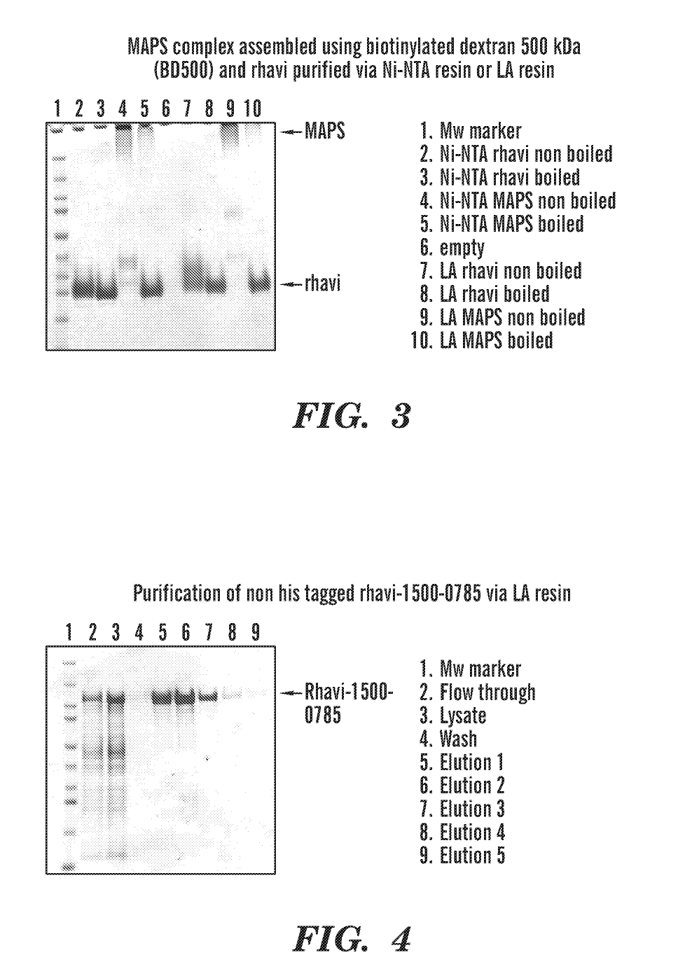

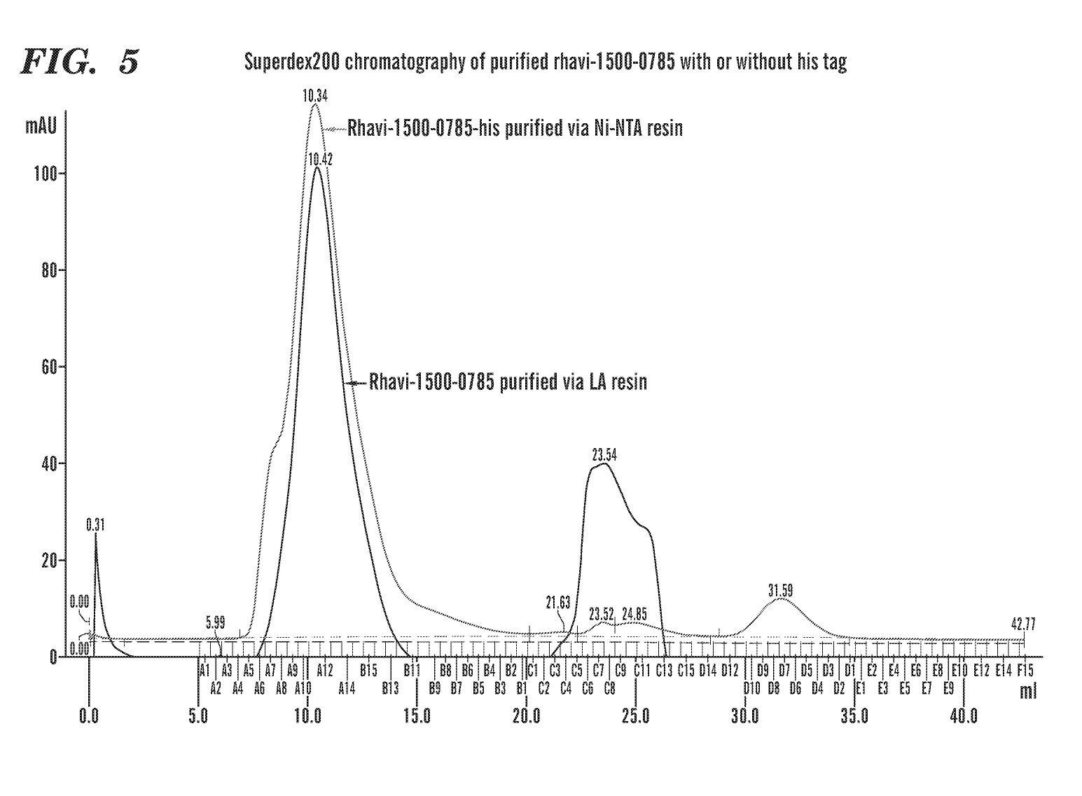

### DETAILED DESCRIPTION

As disclosed herein, one aspect of the present invention relates to methods, kits and compositions for efficient and robust affinity chromatography of a biotin-binding domain, such as a biotin-binding protein, as well as a lipoic acid matrix for such affinity chromatography. In some embodiments, the method relates to the purification of biotin-binding protein of interest, e.g., a rhizavidin protein or fusion protein or complex thereof.

Accordingly, one aspect of the present invention relates to a comprising a lipoic acid (LA) compound immobilized on the surface of a solid support to isolate a biotin-binding protein, such as, for example, a rhizavidin protein or a protein comprising a rhizavidin protein, e.g., a fusion protein or complex comprising a rhizavidin protein. In some embodiments, the lipoic acid (LA) compound is attached to second moiety, e.g., an antibody or bead, and in some embodiments, the antibody or bead can be attached to a solid support. Accordingly, the methods, compositions and kits as disclosed herein enable the separation and isolation of a biotin-binding protein, e.g., a biotin-binding domain, such as, but not limited to, a rhizavidin protein or a protein comprising a rhizavidin protein, such as, e.g., a fusion protein or complex comprising a rhizavidin protein from the rest of the components in the mixture.

One aspect of the present invention relates to a method of reversibly immobilizing a rhizavidin protein or a fusion protein comprising a rhizavidin protein to a support, comprising contacting a rhizavidin protein or a fusion protein comprising a rhizavidin protein to a lipoic acid (LA) compound which is immobilized on a solid support, therefore immobilizing the rhizavidin protein or a fusion protein comprising a rhizavidin protein to the support.

In some embodiments, a rhizavidin protein or a fusion protein comprising a rhizavidin protein is loaded onto the support comprising a lipoic acid (LA) compound immobilized on a solid support in a buffer, e.g., a loading buffer, where the buffer has a pH of between 7.5-9.0. Accordingly, in some embodiments, a rhizavidin protein or a fusion protein comprising a rhizavidin protein is contacted with the solid support in a solution having a pH between 7.5-9.0, for example, where the pH of the solution is about pH 7.5, or about pH 7.9 or about pH 8.0, or about pH 8.2 or about pH 8.5 or about pH 8.7 or about pH 9.0, or anywhere between pH 7.5-9.0. In some embodiments, the solution is an elution buffer.

In some embodiments, to remove the a rhizavidin protein or a fusion protein comprising a rhizavidin protein from the support comprising an immobilized lipoic acid (LA) compound, the support is contacted with an elution buffer having a pH between 8.0-9.5. Accordingly, in some embodiments, the method as disclosed herein further comprises contacting the lipoic acid (LA) compound immobilized on a solid support that also comprises an attached rhizavidin protein or fusion protein comprising a rhizavidin protein with an elution buffer having a pH between 8.0-9.5 In some embodiments, an elution buffer for use in the methods as disclosed herein has a pH of about pH 7.9, or about pH 8.0, or about pH 8.25 or about pH 8.5, or about pH 8.75 or about pH 9.0 or about pH 9.25 or about pH 9.5. In some embodiments, the elution buffer comprises lipoic acid, e.g., 2.5 mg/ml of LA in 20 mM Tris, 1M NaCl, 5% ethanol. In some embodiments, the elution buffer functions to release the biotin-binding protein or a protein comprising a biotin-binding domain, e.g., a rhizavidin protein or a fusion protein comprising a rhizavidin protein from the lipoic acid compound, thereby isolating the rhizavidin protein or a fusion protein thereof into the elution buffer.

This combination of use of a lipoic acid immobilized on a solid support (i.e., a LA-matrix) with the specific wash buffers, removes considerably more impurities than commonly used procedures without damaging the bound biotin-binding protein or affecting recovery. In addition, the disclosed elution conditions and buffers results in a sharper elution peak correlating with a higher concentration of the biotin-binding protein of interest in the eluate, which is advantageous to increase the performance of additional downstream purification processes.

Efficient removal of impurities, including host cell proteins (HCPs) and product-related impurities such as high molecular weight (HMW) species and low molecular weight (LMW) species, is a crucial factor during downstream processing of a biotin-binding protein protein of interest. Affinity chromatography is often used as the first stage of a multi-stage purification process for a biotin-binding protein and the purity of the biotin-binding protein of interest after affinity chromatography notably influences the kind and number of subsequent purification steps. Another important role for affinity chromatography is to concentrate the product, which allows for the use of proportionally smaller, less costly columns in subsequent purification steps. Therefore, it is particularly important to optimize the removal of impurities during the affinity chromatography step.

Low pH conditions, typically between pH 3-4, are a requisite to elute the avidin or streptavidin from a biotin or biotin-derivative affinity matrix and have the drawback of potentially denaturing the avidin or streptavidin and/or inducing aggregation. Accordingly, in some embodiments as disclosed herein, the washing steps and elution steps are performed at a high pH, greater than pH 7.5 or pH 8.0, which preserves the native protein confirmation and secondary and tertiary protein configuration of the biotin-binding protein when bound to the LA-matrix while allowing for removal of impurities, and when the biotin-binding protein is being eluted from the LA-matrix.

In some embodiments, the biotin-binding protein comprises a biotin-binding domain. In some embodiments, the biotin-binding protein is a rhizavidin protein, for example, a rhizavidin protein comprising at least 85%, or at least 87% or at least 89% or at least 90% sequence identity to amino acids of SEQ ID NO: 1.

In some embodiments, the biotin-binding protein is a fusion protein comprising a rhizavidin protein, for example, a fusion protein comprising a rhizavidin protein comprising at least 85%, or at least 87% or at least 89% or at least 90% sequence identity to amino acids of SEQ ID NO: 1, where the fusion protein comprises an additional protein located at the N- and/or C-terminal of the rhizavidin protein.

In some embodiments of the methods as disclosed herein, a solid support to which the lipoic acid is immobilized on, or at the surface, is selected from the group consisting of: plastic, glass, ceramics, silicone, metal, cellulose, beads, membranes and gels or any surface known to an ordinary skilled artisan useful in affinity chromatography. In some embodiments, the solid support is a particle or a magnetic particle or a SEPHAROSE™ bead.

In some embodiments of all aspects as disclosed herein, a lipoic acid compound is bound or linked to the solid support indirectly, e.g., via a protein linker, peptide, nucleic acid, oligosaccharide, glycoprotein or the like. In some embodiments, a lipoic acid compound is bound or linked to the solid support via cross-linking by methods commonly known to persons of ordinary skill in the art.

In some embodiments, a lipoic acid compound for use in the methods, compositions and kits as disclosed herein is lipoic acid or alpha-lipoic acid (ALA), for example, a racemic lipoic acid, or enantiomerically pure or enantiomerically enriched R (+)-alpha-lipoic acid or S-(−)-alpha-lipoic acid. In some embodiments, a lipoic acid compound for use in the methods, compositions and kits as disclosed herein is a lipoic acid derivative, for example, Lipoylpyridoxamine , Lipoylpyridoxamine hydrochloride, Lipoylpyridoxamine hydrobromide, Lipoylpyridoxamine methanesulfonate, Lipoylpyridoxamine p-toluenesulfonate , 1,2-dithiolane analog, diethoxycarbonylated lipoic acid, 6,8-Bisacetylmercaptooctanoic Acid (Bis-acetyl Lipoic Acid), 6,8-Bisbenzoylmercaptooctanoic Acid (Bisbenzoyl Lipoic acid), 8-Acetylmercapto-6-mercaptooctanoic Acid (Monoacetyl Lipoate), 6,8-Biscarbamoylmethylmercaptooctanoic Acid, or other lipoic acid derivatives as disclosed in U.S. Pat. No. 6,331,559, which is incorporated herein in its entirety by reference.

Another aspect of the present invention relates to a kit comprising: (i) a lipoic acid compound attached to a solid support; and (ii) at least one reagent to remove an immobilized rhizavidin protein or fusion protein comprising a rhizavidin protein from the lipoic acid compound attached to the solid support. In some embodiments, the reagent to remove the immobilized rhizavidin protein is an elution buffer comprising a lipoic acid compound as disclosed herein, or a competitive inhibitor of lipoic acid, e.g., biotin or a biotin derivative.

In some embodiments, a kit disclosed herein can further comprise an expression vector comprising the nucleic acid sequence for expression of a Rhziavidin fusion protein, wherein the nucleic acid sequence comprises (i) a nucleic acid sequence encoding a rhizavidin protein comprising at least SEQ ID NO: 1, or a protein of at least 80% sequence identity to SEQ ID NO: 1, and (ii) a nucleic acid comprising a multiple insertion site (MIS) for insertion of a nucleic acid sequence encoding a protein of interest to be fused to the Rhizavidin protein. In some embodiments, the nucleic acid comprising a multiple insertion site (MIS) is at the 5′- of the nucleic acid sequence encoding a rhizavidin protein comprising SEQ ID NO: 1 or a protein of at least 80% sequence identity to SEQ ID NO: 1 such that the protein of interest is at the N-terminus of the rhizavidin protein.

In some embodiments, the expression vector comprises a multiple insertion site (MIS) that is located at the 3′- or the 5′ of the nucleic acid sequence encoding a rhizavidin protein comprising SEQ ID NO: 1 or a protein of at least 80% sequence identity to SEQ ID NO: 1 such that the protein of interest is at the C-terminus of the rhizavidin protein. In some embodiments, the expression vector can optionally further comprise a nucleic acid sequence comprising a linker peptide between the nucleic acid sequence encoding a rhizavidin protein comprising SEQ ID NO: 1 or a protein of at least 80% sequence identity to SEQ ID NO: 1 and the nucleic acid comprising a multiple insertion site (MIS).

In some embodiments, the kit comprises an expression vector for generating a rhizavidin fusion protein comprising a rhizavidin protein that has at least 80% sequence identity to SEQ ID NO: 1 fused to an antigenic peptide or antigen polypeptide, as disclosed herein.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “affinity separation” as used herein refers to a method of separating, purifying, removing, enriching and/or concentrating a component from a mixture or suspension.

The term “fusion protein” as used herein means a protein having at least two elements, one element being a biotin-binding protein and at least a second element, e.g., a protein such as protein antigen or other antigen.

The term “functional derivative” and “mimetic” are used interchangeably, and refers to compounds which possess a biological activity (either functional or structural) that is substantially similar to a biological activity of the entity or molecule for which it's a functional derivative. The term functional derivative is intended to include the fragments, variants, analogues or chemical derivatives of a molecule.

Generally, the biotin-binding protein comprises a biotin-binding domain. As used herein, a “biotin-binding domain” refers to a polypeptide sequence that binds to biotin. While a complete biotin-binding protein can be used as a biotin-binding domain, in some embodiments, only the biotin-binding portion of the protein can be used. In some embodiments, the biotin-binding domain is from Rhizavidin.

The term “biotin-binding” compound as used herein is intended to encompass a compound or protein which is capable of tightly but non-covalently binding to biotin or a biotin derivative. In some embodiments, a biotin-binding compound is Rhizavidin or a fragment thereof. In some embodiments, a biotin-binding compound is Rhizavidin or a fragment thereof which is part of a fusion protein or complex comprising a Rhizavidin or a fragment thereof.

The term “rhizavidin” as used herein refers to the wild type amino acid sequence of rhizavidin as follows:

(SEQ ID NO: 4)
MITT SLYATFGTIADGRRT SGGKTMIRTNAVAALVF AVAT S
ALAFD ASNFKDF S SIAS AS S S WQN
QSGSTMIIQVDSFGNVSGQYVNRAQGTGCQNSPYPLTGRVNGTFIAFSVG
WN STENCNSATG WTGYAQVNGN TEIVTSW
LAYEGGSGPAIEQGQDTFQYVPTTENKSLLKD

In some embodiments, a biotin-binding protein is a fragment of Rhizavidin that lacks the N-terminal amino acids 1-44 of SEQ ID NO: 4, i.e., lacks amino acids MIIT SLYATFGTIADGRRTS GGKTMIRTNAVAALVF AVAT S ALA (SEQ ID NO: 5) of the wild-type of rhizavidin of SEQ ID NO: 4. In some embodiments, a biotin-binding protein is a fragment of Rhizavidin that comprises the amino acid sequence of:

(SEQ ID NO: 1)
FDASNFKDFSSIASASSSWQNQSGSTMIIQVDSFGNVSGQYVNRAQGTG
CQNSPYPLTGRVNGTFIAFSVGWNNSTENCNSATGWTGYAQVNGNNTEI
VTSWNLAYEGGSGPAIEQGQDTFQYVPTTE NKSLLKD.

The terms “lipoic acid” and “alpha lipoic acid” or “α-lipoic acid” are used interchangeably herein and both refer to 1,2-dithione-3-pentanoic acid or 1,2-dithiacyclopentane-3-valeric acid, also known as thioctic acid.

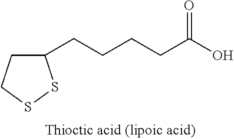 [SMILES: O=C(O)CCCCC1CCSS1]

The term “avidin” as used herein refers to the native egg-white glycoprotein avidin as well as derivatives or equivalents thereof, such as deglycosylated or recombinant forms of avidin, for example, N-acyl avidins, e.g., N-acetyl, N-phthalyl and N-succinyl avidin, and the commercial products ExtrAvidin, Neutralite Avidin and CaptAvidin

The term “Streptavidin” as used herein refers to bacterial streptavidins produced by selected strains of Streptomyces, e.g., Streptomyces avidinii, as well as derivatives or equivalents thereof such as recombinant and truncated streptavidin, such as, for example, “core” streptavidin.

The terms “biotin” as used herein are intended to refer to biotin (cis-hexahydro-2oxo-1H-thieno[3,4]imidazole-4-pentanoic acid) and any biotin derivatives and analogs. Such derivatives and analogues are substances which form a complex with the biotin binding pocket of native or modified streptavidin or avidin. Such compounds include, for example, iminobiotin, desthiobiotin and streptavidin affinity peptides, and also include biotin-.epsilon.-N-lysine, biocytin hydrazide, amino or sulfhydryl derivatives of 2-iminobiotin and biotinyl-ε-aminocaproic acid-N-hydroxysuccinimide ester, sulfo-succinimide-iminobiotin, biotinbromoacetylhydrazide, p-diazobenzoyl biocytin, 3-(N-maleimidopropionyl) biocytin. An some embodiments, a derivative of biotin is desthiobiotin or its derivative DSB-X Biotin, commercially available from Molecular Probes, Eugene, Oreg., USA; product number D20658) (see, US patent Application US 2008/025504, which is incorporated herein in its entirety by reference).

The term “biotinylated substances” or “biotinylated moieties” is to be understood as conjugates of modified biotin or biotin analogues with other moieties such as biomolecules, e.g. nucleic acid molecules (including single or double stranded DNA, RNA, DNA/RNA chimeric molecules, nucleic acid analogs and any molecule which contains or incorporates a nucleotide sequence, e.g. a peptide nucleic acid (PNA) or any modification thereof), proteins (including glycoproteins, enzymes, peptides library or display products and antibodies or derivatives thereof), peptides, carbohydrates or polysaccharides, lipids, etc., wherein the other moieties are covalently linked to the modified biotin or biotin analogues. Many biotinylated ligands are commercially available or can be prepared by standard methods. Processes for coupling a biomolecule, e.g. a nucleic acid molecule or a protein molecule, to biotin are well known in the art (Bayer and Wilchek, Methods in Molec. Biology 10, 143. 1992).

The term “binding partner” is defined as any biological or other organic molecule capable of specific or non-specific binding or interaction with another biological molecule, which binding or interaction may be referred to as “ligand” binding or interaction and is exemplified by, but not limited to, antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector or repressor/inducer bindings or interactions. The appropriate ligands will be chosen depending on the use to which the method of the invention is desired to be put.

In some instances, the ligand is an antibody which is directed against a drug, hormone, antibiotic or other compound having antigenic properties. The antibody may also be directed against another antibody (that is, an anti-antibody). Both monoclonal and polyclonal antibodies can be used, and they can be whole molecules or various fragments thereof. Antibody specific for a particular ligand may be produced by methods well known and documented in the art.

Antibodies for use in methods of the present invention may be of any species, class or subtype providing that such antibodies are capable of forming a linkage with a particular target ligand and can be biotinylated with a modified biotin. Thus antibodies for use in the present invention include: any of the various classes or sub-classes of immunoglobulin, e.g. IgG, IgA, IgM, IgD or IgE derived from any animal e.g. any of the animals conventionally used, e.g. sheep, rabbits, goats, or mice, monoclonal antibodies, intact antibodies or “fragments” of antibodies, monoclonal or polyclonal, the fragments being those which contain the binding region of the antibody, e.g. fragments devoid of the Fc portion (e.g. Fab, Fab′, F(ab′)2, Fv), the so called “half molecule” fragments obtained by reductive cleavage of the disulphide bonds connecting the heavy chain components in the intact antibody, antibodies produced or modified by recombinant DNA or other synthetic techniques, including monoclonal antibodies, fragments of antibodies, “humanized antibodies”, chimeric antibodies, or synthetically made or altered antibody-like structures. Also included are functional derivatives or “equivalents” of antibodies e.g. single chain antibodies.

Alternatively, the ligand can be an antigenic material (including mono- or multivalent or multi-determinant antigens).

The terms “conjugate” and “complex” as used herein refer to any conjugate or complex comprising a biotin-binding domain protein, present as a protein or a fusion protein, or linked to another entity by covalent (e.g., a peptide bond) or non-covalent bonding Typically, a biotin-binding domain, e.g., a rhizavidin protein can be bound or linked to one or more, preferably one, biological or chemical entity, e.g., a biomolecule, or other protein such as an antigen.

The terms “reversal”, “cleaving”, “releasing”, or “disrupting” are used herein interchangeably and are intended to mean physical separation or detachment or dissociation of the partners of the binding complex. What is required, is that the linkage between the lipoic acid compound and the biotin-binding domain, e.g., rhizavidin is disrupted or broken to allow separation of the respective entities.

The “displacement molecule” (for example, a lipoic acid compound) may physically break or destabilize the linkage between the biotin-binding domain (e.g., a rhizavidin protein) and the lipoic acid compound in a sufficient manner to allow it to be cleaved, or reversed, thus allowing the two linked entities to be separated. Furthermore, in a population of linkages, it may not be necessary for each and every linkage to be disrupted, as long as a sufficient or significant proportion are “reversed” e.g. where substantially all of the linkages are “reversed”. “Substantially” in this context, may be taken to mean that at least 70% (or more preferably at least 75, 80, 85, 90 or 95%) of the linkages are reversed. Ideally, 100% of the linkages are reversed. In the linkage reversal system of the present invention, utility may be preserved even though reversal may not be 100% complete.

The term “derivative” as used herein refers to proteins or peptides (e.g., rhizavidin proteins or fusion proteins thereof) which have been chemically modified, for example but not limited to by techniques such as ubiquitination, labeling, pegylation (derivatization with polyethylene glycol) or addition of other molecules.

As used herein, “variant” with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide). A “variant” of a rhizavidin protein for example, is meant to refer to a molecule substantially similar in structure and function, i.e. where the function is the ability to bind to biotin or a biotin derivative, or to a lipoic acid compound as disclosed herein. A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical.

For example, a variant of a rhizavidin protein can contain a mutation or modification that differs from a reference amino acid of SEQ ID NO: 1. In some embodiments, a variant can be a different isoform of a rhizavidin protein or can comprise different isomer amino acids. Variants can be naturally-occurring, synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. the ability of the peptide to penetrate the BBB). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In some embodiments, individual substitutions, deletions or additions that alter, add or delete a single amino acid or a small percentage of amino acids can also be considered “conservative substitutions” is the change does not reduce the activity of the peptide (i.e. the ability of, for example MIS to bind and activate MISRII). Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents. As used herein, the term “nonconservative” refers to substituting an amino acid residue for a different amino acid residue that has different chemical properties. The nonconservative substitutions include, but are not limited to aspartic acid (D) being replaced with glycine (G); asparagine (N) being replaced with lysine (K); or alanine (A) being replaced with arginine (R).

“Insertions” or “deletions” are typically in the range of about 1 to 5 amino acids. The variation allowed can be experimentally determined by producing the peptide synthetically while systematically making insertions, deletions, or substitutions of nucleotides in the sequence using recombinant DNA techniques.

The term “functional derivative” and “mimetic” are used interchangeably, and refers to a compound which possess a biological activity (either functional or structural) that is substantially similar to a biological activity of the entity or molecule its is a functional derivative of The term functional derivative is intended to include the fragments, variants, analogues or chemical derivatives of a molecule.

A “fragment” of a molecule, is meant to refer to any contagious polypeptide subset of the molecule. Fragments of, for example a rhizavidin protein which have the same activity as that of amino acid of SEQ ID NO: 1 are also encompassed for use in the present invention.

An “analog” of a molecule such as a rhizavidin protein, for example an analogue of the protein of amino acid of SEQ ID NO: 1 is meant to refer to a molecule similar in function to either the entire molecule or to a fragment thereof of SEQ ID NO: 1. As used herein, a molecule is said to be a “chemical derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., Mack Publ., Easton, Pa. (1990).

As used herein, “homologous”, when used to describe a polypeptide or polynucleotide, indicates that two polypeptides or two polynucleotides, or designated sequences thereof, when optimally aligned and compared, are identical, with appropriate amino acid or nucleotide insertions or deletions, in at least 70% of the amino acids or nucleotides, usually from about 75% to 99%, and more preferably at least about 98 to 99% of the amino acids or nucleotides.

The term “homolog” or “homologous” can also be used with respect to structure and/or function. With respect to amino acid sequence homology, amino acid sequences are homologs if they are at least 50%, at least 60 at least 70%, at least 80%, at least 90%, at least 95% identical, at least 97% identical, or at least 99% identical. The term “substantially homologous” refers to sequences that are at least 90%, at least 95% identical, at least 97% identical or at least 99% identical. Homologous sequences can be the same functional gene in different species.

As used herein, the term “substantial similarity” in the context of polypeptide sequences, indicates that the polypeptide comprises a sequence with at least 60% sequence identity to a reference sequence, or 70%, or 80%, 85% or 87% sequence identity to the reference sequence, or most preferably 90% identity over a comparison window of about 10-20 amino acid residues. In some embodiments, a rhizavidin protein with substantial similarity to SEQ ID NO: 1 is a rhizavidin protein that has at least about 70%, or about 80%, or about 85% or about 87% or about 90% or more sequence identity to SEQ ID NO: 1, and can have a similar biological function or activity, e.g., at least 80% binding ability to biotin as compared to the rhizavidin protein of SEQ ID NO: 1.

In the context of amino acid sequences, “substantial similarity” further includes conservative substitutions of amino acids. Thus, a polypeptide is substantially similar to a second polypeptide, for example, where the two peptides differ by one or more conservative substitutions. The term “substantial identity” means that two peptide sequences, when optimally aligned, such as by the programs GAP or BESTFIT using default gap weights, share at least 65 percent sequence identity, preferably at least 80 or 90 percent sequence identity, more preferably at least 95 percent sequence identity or more (e.g., 99 percent sequence identity or higher). Preferably, residue positions which are not identical differ by conservative amino acid substitutions.

Determination of homologs of the genes or peptides of the present invention can be easily ascertained by the skilled artisan. The terms “homology” or “identity” or “similarity” are used interchangeably herein and refers to sequence similarity between two peptides or between two nucleic acid molecules. Homology and identity can each be determined by comparing a position in each sequence which can be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application.

In one embodiment, the term “rhizavidin homolog” refers to an amino acid sequence that has 40% homology to the SEQ ID NO: 1 as disclosed herein, or more preferably at least about 50%, still more preferably, at least about 60% homology (i.e., sequence identity), still more preferably, at least about 70% homology, even more preferably, at least about 75% homology, yet more preferably, at least about 80% homology, even more preferably at least about 85% homology, still more preferably, at least about 90% homology, and more preferably, at least about 95% homology (or sequence identity) to SEQ ID NO: 1. As discussed above, the homology is at least about 50% to 100% and all intervals in between (i.e., 55%, 60%, 70%, 75%, 80%, 85%, 90%, 95%, 98%, etc.).

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=4, and a comparison of both strands.

The term “analog” as used herein, is indented to include allelic, species and induced variants. Analogs typically differ from naturally occurring peptides at one or a few positions, often by virtue of conservative substitutions. Analogs typically exhibit at least 80 or 90% sequence identity with natural peptides or polypeptides (e.g., SEQ ID NO: 1). Some analogs also include unnatural amino acids or modifications of N or C terminal amino acids. Examples of unnatural amino acids are, for example but not limited to; acedisubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine. Fragments and analogs can be screened for prophylactic or therapeutic efficacy in transgenic animal models as described below.

The term “substitution” when referring to a peptide, refers to a change in an amino acid for a different entity, for example another amino acid or amino-acid moiety. Substitutions can be conservative or non-conservative substitutions.

The term “substantially pure”, with respect to the isolation of a biotin-binding domain, e.g., a rhizavidin protein as disclosed herein, refers to a sample that is at least about 65%, or at least about 75%, or at least about 85%, or at least about 90%, or at least about 95% pure, with respect to the biotin-binding domain (e.g., rhizavidin protein) as compared to the total protein concentration in the sample. Stated another way, the terms “substantially pure” or “essentially purified”, with regard to a preparation of a biotin-binding domain, (e.g., a rhizavidin protein) isolated and purified using the lipoic acid compound matrix as disclosed herein, refer to a protein sample that contain fewer than about 20%, more less than about 15%, 10%, 8%, 7%, or fewer than about 5%, 4%, 3%, 2%, 1%, or less than 1%, of non-biotin binding domains (e.g., a rhizavidin protein).

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The articles “a” and “an” are used herein to refer to one or to more than one (i.e., to at least one) of the grammatical object of the article. By way of example, “an element” means one element or more than one element.

While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

### Methods of Isolating a Rhizavidin Protein or a Complex or Fusion Protein Comprising the Same.

### Lipoic Acid (LA) Compounds

In some embodiments, a lipoic acid compound for use in the methods, compositions and kits as disclosed herein is α-lipoic acid (also known as thioctic acid) having the following structure:

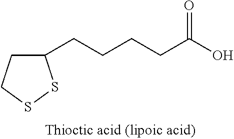 [SMILES: O=C(O)CCCCC1CCSS1]

Lipoic acid (LA), also known as α-lipoic acid and alpha lipoic acid (ALA) and thioctic acid and is an organosulfur compound derived from octanoic acid. LA contains two sulfur atoms (at C6 and C8) connected by a disulfide bond and is thus considered to be oxidized although either sulfur atom can exist in higher oxidation states. The carbon atom at C6 is chiral and the molecule exists as two enantiomers (R)-(+)-lipoic acid (RLA) and (S)-(−)-lipoic acid (SLA) and as a racemic mixture (R/S)-lipoic acid (R/S-LA). LA appears physically as a yellow solid and structurally contains a terminal carboxylic acid and a terminal dithiolane ring.

In some embodiments, a lipoic acid compound for use in the methods, compositions and kits as disclosed herein is α-dihydrolipoic acid the following structure, where * represents the chiral center:

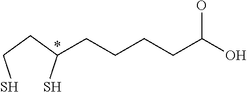 [SMILES: OC(O)CCCCC(S)CCS]

Lipoic acid exists as two enantiomers: the R-enantiomer and the S-enantiomers. Naturally-occurring lipoic acid occurs in the R-form, but synthetic lipoic acid (commonly known as alpha lipoic acid) is a racemic mixture of R-form and S-form. It should be understood that throughout the application, reference to lipoic acid therefore includes the R-enantiomer, the S-enantiomer, and the racemic mixture of R/S enantiomers.

In some embodiments, the lipoic acid is the R-form. In some embodiments, the lipoic acid is the S-form. In some embodiments where the lipoic acid is in the S-form, the (S)-lipoic acid does not need a sulfo-NHS linker to attach/couple the (S)-lipoic acid the resin or solid support, but rather can be directly attached to a solid support. In some embodiments, the LA-resin or LA-matrix comprises any one of; R-form lipoic acid, S-form of lipoic acid or both (i.e., racemic mixture of the R- and S-form of the lipoic acid). For example, the (R)-lipoic acid (also referred to as RLA) is shown in the top, and the (S)-lipoic acid (also referred to as SLA) is shown below.

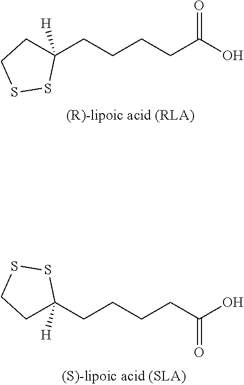 [SMILES: O=C(O)CCCC[C@@H]1CCSS1 ; O=C(O)CCCC[C@H]1CCSS1]

A 1:1 mixture (racemate) of (R)- and (S)-lipoic acid is called (RS)-lipoic acid or (±)-lipoic acid (R/S-LA). In some embodiments, a lipoic acid compound or component for use in the methods, compositions and kits as disclosed herein is a racemic or racemate mixture of any of α-lipoic acid, enantiopure R-(+)- or S-(−)-α-lipoic acid or any mixtures thereof, as well as racemic α-dihydrolipoic acid (6,8-dimercaptooctanoic acid or DHLA), enantiopure R-(−)- or S-(+)-dihydrolipoic acid or any mixtures thereof. Also encompassed in some embodiments is the use of α-lipoic acid or dihydrolipoic acid as such or wholly or partly in the form of their salts such as, for example, creatine, sodium, potassium, ammonium or ornithine lipoates for the production of the solution. The production of racemic a-lipoic acid, of enantiopure or enantiomer-enriched R-(+)- or S-(−)-α-lipoic acid, of racemic dihydrolipoic acid, enantiopure or enantiomer-enriched R-(−)- or S-(+)-dihydrolipoic acid and of salts or mixtures thereof can take place in a known manner. The preparation of racemic α-lipoic acid, enantiomerically pure or enantiomerically enriched R (+)- or S-(−)-α-lipoic acid, racemic dihydrolipoic, enantiomerically pure or enantiomerically enriched R-(−)- or S (+)-Dihydroliponsaure and of their salts or mixtures can be carried out in a known manner, for example, as disclosed in WO2003047567 or US Application 20040266858, and U.S. Pat. Nos. 5,728,735, 5,281,722, 6,271,254, and 5,650,429, which are incorporated herein in their entirety by reference.

In some embodiments, the a lipoic acid compound or component for use in the methods, compositions and kits as disclosed herein is a lipoic acid derivative, as disclosed in U.S. Pat. Nos. 3,288,797 and 6,331,559 which are incorporated herein in their entirety by reference.

As discussed above, α-lipoic acid or α-dihydrolipoic acid can be in a S- or R-form, or exist as a racemic (R- and S-) mixture of α-lipoic acid or α-dihydrolipoic acid. In some embodiments, the solid support comprises both α-lipoic acid and α-dihydrolipoic acid, either in the S- or R-form, or as a racemic (R- and S-) mixture of α-lipoic acid or α-dihydrolipoic acid.

In some embodiments, the lipoic acid compound is a lipoic acid derivative known in the art. Such lipoic acid derivatives encompassed for use in the methods, compositions and kits as disclosed herein, include, but are not limited to, e.g., Lipoylpyridoxamine, Lipoylpyridoxamine hydrochloride, Lipoylpyridoxamine hydrobromide, Lipoylpyridoxamine methanesulfonate, Lipoylpyridoxamine p-toluenesulfonate, 1,2-dithiolane analog, diethoxycarbonylated lipoic acid, 6,8-Bisacetylmercaptooctanoic Acid (Bis-acetyl Lipoic Acid), 6,8-Bisbenzoylmercaptooctanoic Acid (Bisbenzoyl Lipoic acid), 8-Acetylmercapto-6-mercaptooctanoic Acid (Monoacetyl Lipoate), 6,8-Biscarbamoylmethylmercaptooctanoic Acid, 6,8-Bis-[S—(N-methylsuccinimido)]mercaptooctanoic Acid, as disclosed in U.S. Pat. No. 6,331,559, which is incorporated herein in its entirety by reference.

In some embodiments, a lipoic acid derivative is a class of compounds comprise the structure of formula I:

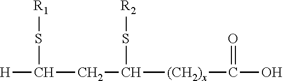

wherein:

x is 0-16

R₁and R₂ are independently acyl R₃C(O); wherein R₃ is an alkyl or aryl group; alkyl CₙH₂ₙ₊₁; alkenyl CₘH₂ₘ₋₁; alkynyl CₘH₂ₘ₋₃; aryl, alkyl sulfide CH₃(CH₂)ₙ—S—; imidoyl CH₃(CH₂)ₙC(═NH)—; and semiacetal R₄CH(OH)—S—; wherein R₄ is CCl₃ or COOH; and wherein n is 0-10 and m is 2-10.

In another embodiment, a lipoic acid derivative is a class of compounds the structure of formula II:

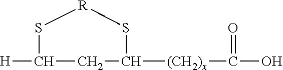

x is 0-16; and

R is a non-palladium metal chelate.

One or both of the thiol portions of the lipoic acid composition may be altered or complexed (i.e., derivatized) with an additional reagent or moiety. In some embodiments, a lipoic acid derivative for use in the methods, kits and compositions as disclosed herein will vary according to the type of biotin-binding protein to be purified.

### Cross-Linking Reagents and Cross Linking the LA Compound to a Solid Support

In some embodiments, lipoic acid is immobilized to a solid support as disclosed in Mahlicli et al., “Immobilization of alpha lipoic acid onto polysulfone membranes to suppress hemodialysis induced oxidative stress,” J. Membrane Sci., 2014; 449; 27-37; which is disclosed herein in its entirety by reference.

In some embodiments, lipoic acid is immobilized to a solid support as disclosed Harmon et al., “Purification of antibodies against biotin on lipoic acid-sepharose”, Analytical Biochemistry, 1980; 103 (1), 58-63, and also disclosed in Ryan et al., J. General Microbiology, “The Isolation of Rhodanese from Pseudomonas aeruginosa by Affinity Chromatography” General Microbiology, 1977; 103; 197-199, which are disclosed herein in their entirety by reference.

In some embodiments, the lipoic acid compound is cross-linked to the solid support with a cross-linking reagent, for example, a cross-linking reagent selected from CDAP(1-cyano-4-dimethylaminopyridinium tetrafluoroborate), EDC (1-Ethyl-3-[3-dimethylaminopropyl] carbodiimide hydrochloride), sodium cyanoborohydride; cyanogen bromide; or ammonium bicarbonate/iodoacetic acid. In some embodiments, the lipoic acid compound is cross-linked to carboxyl, hydroxyl, amino, phenoxyl, hemiacetal, and mecapto functional groups of the solid support. In some embodiments, the lipoic acid compound is covalently bonded to the solid support.

Many bivalent or polyvalent linking agents are useful in coupling molecules to other molecules. For example, representative coupling agents can include organic compounds such as thioesters, carbodiimides, succinimide esters, disocyanates, glutaraldehydes, diazobenzenes and hexamethylene diamines. This listing is not intended to be exhaustive of the various classes of coupling agents known in the art but, rather, is exemplary of the more common coupling agents. See Killen & Lindstrom, 133 J. Immunol. 1335 (1984); Jansen et al., 62 Imm. Rev. 185 (1982); Vitetta et al.

In some embodiments, cross-linking reagents agents described in the literature are encompassed for use in the methods, immunogenic compositions and kits as disclosed herein. See, e.g., Ramakrishnan, et al., 44 Cancer Res. 201 (1984) (describing the use of MBS (M-maleimidobenzoyl-N-hydroxysuccinimide ester)); Umemoto et al., U.S. Patent No. 5,030,719 (describing the use of a halogenated acetyl hydrazide derivative coupled to an antibody by way of an oligopeptide linker). Particular linkers include: (a) EDC (1-ethyl-3-(3-dimethylamino-propyl) carbodiimide hydrochloride; (b) SMPT (4-succinimidyloxycarbonyl-alpha-methyl-alpha-(2-pyridyl-dithio)-toluene (Pierce Chem. Co., Cat. (21558G); (c) SPDP (succinimidyl-6 [3-(2-pyridyldithio) propionamido] hexanoate (Pierce Chem. Co., Cat #21651G); (d) Sulfo-LC-SPDP (sulfosuccinimidyl 6 [3-(2-pyridyldithio)-propianamide] hexanoate (Pierce Chem. Co. Cat. #2165-G); and (f) sulfo-NHS (N-hydroxysulfo-succinimide: Pierce Chem. Co., Cat. #24510) conjugated to EDC.

The linkages or linking agents described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage can be cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments, the lipoic acid is cross-linked to the support using any of the cross-linkers, CDAP (1-cyano-4-dimethylaminopyridinium tetrafluoroborate), EDC (1-Ethyl-3-[3-dimethylaminopropyl] carbodiimide hydrochloride), sodium cyanoborohydride, cyanogen bromide, or ammonium bicarbonate/iodoacetic acid or derivatives thereof.

Exemplary cross-linking molecules for use in the methods and immunogenic compositions as disclosed herein include, but are not limited to those listed in Tables 1 and 2.

TABLE 1
Exemplary homobifunctional crosslinkers. Homobifunctional
crosslinkers are crosslinking reagents that have the same
type of reactive group at either end. Reagents are classified
by what chemical groups they cross link (left column) and
their chemical composition (middle column). Products are listed
in order of increasing length within each cell.

| Cross- | | |
|---|---|---|
| linking | Crosslinker Reactive | |
| Target | Groups, Features | Example Products |
| Amine-to- | NHS esters | DSG; DSS; BS3; TSAT |
| Amine | | (trifunctional); Bioconjugate |
| | | Toolkit Reagent Pairs |
| | NHS esters, PEG spacer | BS(PEG)5; BS(PEG)9 |
| | NHS esters, thiol- | DSP; DTSSP |
| | cleavable | |
| | NHS esters, misc- | DST; BSOCOES; EGS; |
| | cleavable | Sulfo-EGS |
| | Imidoesters | DMA; DMP; DMS |
| | Imidoesters, thiol- | DTBP |
| | cleavable | |
| | Other | DFDNB; THPP (trifunctional); |
| | | Aldehyde-Activated Dextran Kit |
| Sulfhydryl- | Maleimides | BMOE; BMB; BMH; |
| to-Sulfhydryl | | TMEA (trifunctional) |
| | Maleimides, PEG spacer | BM(PEG)2; BM(PEG)3 |
| | Maleimides, cleavable | BMDB; DTME |
| | Pyridyldithiols | DPDPB |
| | (cleavable) | |
| | Other | HBVS (vinylsulfone) |
| Nonselective | Aryl azides | BASED (thiol-cleavable) |

TABLE 2
Exemplary heterobifunctional crosslinkers. Heterobifunctional
crosslinkers are crosslinking reagents that have the different
reactive groups at either end. Reagents are classified by
what chemical groups they cross link (left column) and their
chemical composition (middle column). Products are listed
in order of increasing length within each cell.

| Cross- | | |
|---|---|---|
| linking | Crosslinker Reactive | |
| Targets | Groups, Features | Example Products |
| Amine-to- | NHS ester/Maleimide | AMAS; BMPS; GMBS and |
| Sulfhydryl | | Sulfo-GMBS; MBS and Sulfo- |
| | | MBS; SMCC and Sulfo-SMCC; |
| | | EMCS and Sulfo-EMCS; SMPB |
| | | and Sulfo-SMPB; SMPH; LC- |
| | | SMCC; Sulfo-KMUS |
| | NHS ester/Maleimide, | SM(PEG)2; SM(PEG)4; |
| | PEG spacer | SM(PEG)6; SM(PEG)8; |
| | | SM(PEG)12; SM(PEG)24 |
| | NHS ester/ | SPDP; LC-SPDP and Sulfo-LC- |
| | Pyridyldithiol, | SPDP; SMPT; Sulfo-LC-SMPT |
| | cleavable | |
| | NHS esters/Haloacetyl | SIA; SBAP; SIAB; Sulfo-SIAB |
| Amine-to- | NHS ester/Aryl Azide | NHS-ASA |
| Nonselective | | ANB-NOS |
| | | Sulfo-HSAB |
| | | Sulfo-NHS-LC-ASA |
| | | SANPAH and Sulfo-SANPAH |
| | NHS ester/Aryl Azide, | Sulfo-SFAD; Sulfo-SAND; |
| | cleavable | Sulfo-SAED |
| | NHS ester/Diazirine | SDA and Sulfo-SDA; LC-SDA |
| | | and Sulfo-LC-SDA |
| | NHS ester/Diazirine, | SDAD and Sulfo-SDAD |
| | cleavable | |
| Amine-to- | Carbodiimide | DCC; EDC |
| Carboxyl | | |
| Sulfhydryl-to- | Pyridyldithiol/ | APDP |
| Nonselective | Aryl Azide | |
| Sulfhydryl-to- | Maleimide/Hydrazide | BMPH; EMCH; MPBH; KMUH |
| Carbohydrate | Pyridyldithiol/ | BMPH; EMCH; MPBH; KMUH |
| | Hydrazide | |
| Carbohydrate- | Hydrazide/Aryl Azide | ABH |
| to-Nonselective | | |
| Hydroxyl-to- | Isocyanate/Maleimide | PMPI |
| Sulfhydryl | | |
| Amine-to-DNA | NHS ester/Psoralen | SPB |

Activated/Functionalized Matrices

In some embodiments, the disclosure herein relates to the production of a LA-matrix using pre-activated supports. For example, Sigma offers a variety of activated matrices ready for direct coupling of a LA compound as disclosed herein. The major properties for each direct activation type are described in the accompanying Table 3.

TABLE 3
Direct activated matrices.

| | | | | | Bond | |
|---|---|---|---|---|---|---|
| | Linkage | Available | | | Type; | |
| | to | Reactive | Specificity | Reaction | Stability of | Intrinsic |
| Activation | Resin | Group | of Group | Conditions | Attachment | Spacer |
| Carbonyl- | Carbamate | Imidazole | Amine | pH 8-10 | Carbamate; | 1 atom |
| dimidazole | | Carbamate | | | good | neutral |
| | | | | | stability | |
| | | | | | below | |
| | | | | | pH 10 | |
| Cyanogen | Final | Cyanate | Amine | pH 8-9.5 | Isourea; | 1 atom |
| Bromide | isourea | ester | | | moderately | cationic |
| | | | | | stable | |
| Epichlorohydrin | Ether | Epoxy | SH > NH₂ > | pH 7-8 SH | Thioether | 3 atoms |
| | | | OH | pH 9-11 | sec amine | neutral |
| | | | | NH₂ | ether; all | |
| | | | | pH > 11 | very stable | |
| | | | | OH | | |
| Epoxy (bis) | Ether | Epoxy | SH > NH₂ > | pH 7-8 SH | Thioether | 12 |
| | | | OH | pH 9-11 | sec amine | atoms |
| | | | | NH₂ | ether; all | neutral |
| | | | | pH > 11 | very stable | |
| | | | | OH | | |
| N-Hydroxy- | Final | Succinimidyl | Amine | pH 8-9.5 | Carbamate; | 1 atom |
| succinimidyl | carbonate | Carbonate | | | good | neutral |
| Chloroformate | | | | | stability | |
| | | | | | below | |
| | | | | | pH 10 | |
| p-Nitrophenyl | Final | Nitrophenyl | Amine | pH 8.5-10 | Carbomate; | 1 atom |
| Chloroformate | carbonate | Carbonate | | | good | neutral |
| | | | | | stability | |
| | | | | | below | |
| | | | | | pH 10 | |
| Tresyl Chloride | Alkylamine | Tresyl | Amine, thiol | pH 7-9.5 | Alkylamine | 0 atoms |
| | thioether | sulfonate | | | thioether; | direct |
| | | | | | both very | linkage |
| | | | | | stable | |
| Vinyl Sulfone | Sulfonyl | Vinyl | SH > NH₂ > | pH 6-8 SH | ThiotherSec | 5 atoms |
| | thioether | sulfone | OH | pH 8-10 | amine | neutral, |
| | | | | NH₂ | ether; Good | some |
| | | | | pH > 10 | stability | non- |
| | | | | OH | below pH 9 | specific |
| | | | | | | effects |

In some embodiments, the solid support can be an activated matrix which contain an additional spacer due to prior derivatization. These resins are suitable for the present invention as they allow milder coupling conditions or a more specific attachment of a ligand. Examples of these are matrices are shown in Table 4. In some embodiments, matrices with incorporated terminal groups suitable for custom derivatization or coupling. Typically these groups will be a carboxyl or amine function, which may be coupled by an amide bond to the lipoic acid compound, and require an additional reagent to accomplish condensation. Another group commonly utilized is hydrazide, which may couple to aldehydes by hydrazone formation and typically does not require additional reagent.

TABLE 4
Activated Groups Incorporated to Prederivatized Matrices

| | Speci- | Coupling | Bond to ligand; |
|---|---|---|---|
| Active Group | ficity | Conditions | stability |
| N-Hydroxysuccinimide | Amine | pH 6.0-8.0 | Amine; good stability |
| Ester; active ester | | | |
| Disulfide; reactivity | Sulf- | pH 6.0-8.0 | Covalent disulfide; |
| based on leaving group | hydryl | | good stability under |
| | | | nonreducing conditions |

### Solid Supports

In some embodiments, a lipoic acid (LA) compound is immobilized on an immobilizing moiety, e.g. a solid support. Such a solid support comprising an immobilized lipoic acid compound is also referred to herein as a “LA-matrix”. In some embodiments, a lipoic acid (LA) compound will be immobilized directly on the surface of a solid support, and in alternative embodiments, a lipoic acid (LA) compound can be attached to on an intermediate entity, e.g., an antibody or bead, and the intermediate entity, e.g., antibody or bead, can be immobilized on a solid support. The attachment of either component of the linkage to a solid phase allows easy manipulation of the linked components. Thus, the attachment to some kind of solid phase can enable the separation of the linked components from the rest of the components in the mixture. This can be achieved for example by carrying out washing steps, or if the components are attached to magnetic beads, using a magnetic field to effect physical separation of the linked component from the rest of the components in the mixture.

As used herein, the term “lipoic acid matrix” or “LA matrix”, is intended to refer to a solid phase medium, typically a gel or resin, that allows for separation of biochemical mixtures based on a highly specific binding interaction between a protein of interest (e.g., a biotin-binding protein, such as a rhizavidin protein as disclosed herein) and the LA matrix. Thus, the solid phase medium comprises a lipoic acid compound to which the protein of interest (e.g., a biotin-binding protein, such as a rhizavidin protein as disclosed herein) is capable of reversibly affixing, depending upon the buffer conditions. Non-limiting examples of immobilized or solid phase media that can comprise the LA matrix include a gel matrix, such as agarose beads (such as commercially available Sepharose or SEPHAROSE™ matrices), and a glass matrix, such as porous glass beads (such as commercially available ProSep matrices).

In some embodiments, a lipoic acid matrix as disclosed herein is a reusable LA matrix, for example, it can be used for purification of a biotin-binding protein (e.g., rhizavidin protein or fusion protein thereof) more than once, for example, but not limited to, at least 2, or at least 3, or at least 4, or at least 5, or at least 6, or at least 7, or at least 8, or at least 9, or at least 10, or between 10-15, or between 15-20 or more than 20 times, but less than 50 times.

The solid support may be any of the well-known supports or matrices which are currently widely used or proposed for immobilization, separation etc., in chemical or biochemical procedures. These may take the form of particles, sheets, dip-sticks, gels, filters, membranes, microfibre strips, tubes, wells or plates, fibers or capillaries, combs, pipette tips, microarrays or chips or combinations thereof, and conveniently may be made of a polymeric material, e.g. agarose, SEPHAROSE™, cellulose, nitrocellulose, alginate, Teflon, latex, acrylamide, nylon membranes, plastic, polystyrene, glass or silica or metals. Biochips may be used as solid supports to provide miniature experimental systems as described for example in Nilsson et al. (Anal. Biochem. 224: 400-408, 1995) or as a diagnostic tool. Numerous suitable solid supports are commercially available.

In some embodiments, a solid support can be a matrix or affinity chromatography matrix (also referred to herein as “AC matrix”) and is a Protein A column. In various other embodiments, the matrix can be, for example, selected from the group consisting of a Protein G column, a Protein A/G column, a Protein L column, an immobilized metal ion affinity chromatography (IMAC) column, a calmodulin resin column, a MEP HYPERCEL™ column, a column that binds maltose binding protein (MBP), a column that binds glutathione-S-transferase (GST) and a column that binds Strep-Tag II.

As used herein, the term “affinity chromatography matrix” or “AC matrix”, is intended to refer to a solid phase medium, typically a gel or resin, that allows for separation of biochemical mixtures based on a highly specific binding interaction between a protein of interest (e.g., the biotin-binding protein, e.g., rhizavidin protein comprising SEQ ID NO: 1 or at least 80% sequence identity thereto) and the AC matrix. Thus, the solid phase medium comprises a target to which the protein of interest is capable of reversibly affixing, depending upon the buffer conditions. Non-limiting examples of immobilized or solid phase media that can comprise the AC matrix include a gel matrix, such as agarose beads (such as commercially available Sepharose or SEPHAROSE™ matrices), and a glass matrix, such as porous glass beads (such as commercially available ProSep matrices).

Binding of the protein of interest (e.g., the biotin-binding protein, e.g., rhizavidin protein comprising SEQ ID NO: 1 or at least 80% sequence identity thereto) to the AC matrix typically is achieved by column chromatography. That is, the AC matrix is formed into a column, a biochemical mixture containing the biotin-binding protein, e.g., rhizavidin protein comprising SEQ ID NO: 1 or at least 80% sequence identity thereto, or a fusion protein thereof, is flowed through the column, followed by washing of the column by flowing through the column one or more wash solutions, followed by elution of the protein of interest from the column by flowing through the column an elution buffer.

Alternatively, binding of the protein of interest (e.g., a biotin-binding protein, e.g., rhizavidin protein comprising SEQ ID NO: 1 or at least 80% sequence identity thereto, or fusion protein thereof) to the AC matrix can be achieved by batch treatment, in which the biochemical mixtures containing the protein of interest is incubated with the AC matrix in a vessel to allow for binding of the protein of interest to the AC matrix, the solid phase medium is removed from the vessel (e.g., by centrifugation), the solid phase medium is washed to remove impurities and again recovered (e.g., by centrifugation) and the protein of interest is eluted from the solid phase medium.

In yet another embodiment, a combination of batch treatment and column chromatography can be used. For example, the initial binding of the protein of interest to the AC matrix can be achieved by batch treatment and then the solid phase medium can be packed into a column, following by washing of the column and elution of the protein of interest from the column.

In some embodiments, a lipoic acid (LA) compound is immobilized directly on the surface of a solid support AC matrix that is a Protein A column, which comprises as the target attached to the solid phase a bacterial cell wall protein, Protein A.

Various Protein A resins are well known in the art and suitable for use in the invention. Non-limiting examples of commercially available Protein A resins include MabSelect, MabSelect Xtra, MabSelect Sure, nProtein A Sepharose FF, rmpProtein A Sepharose FF, Protein A Sepharose CL-4B and nProtein A Sepharose 4 FF (all commercially available from GE Healthcare); ProSep A, ProSep-vA High Capacity, ProSep-vA Ultra and ProSep-Va Ultra Plus (all commercially available from Millipore); Poros A and Mabcapture A (both commercially available from Poros); IPA-300, IPA-400 and IPA-500 (all commercially available from RepliGen Corp.); Affigel protein A and Affiprep protein A (both commercially available from Bio-Rad); MABsorbent AlP and MABsorbent A2P (both commercially available from Affinity Chromatography Ltd.): Protein A Ceramic Hyper D F (commercially available from Pall Corporation); Ultralink Immobilized protein A and Agarose protein A (both commercially available from PIERCE) and Protein A Cell thru 300 and Protein A Ultraflow (both commercially available from Sterogen Bioseparations).

In addition to Protein A chromatography, the washing method of the invention can be applied to other affinity chromatography systems. For example, in another embodiment, the LA matrix can be a Protein G column, a Protein A/G column or a Protein L column. Thus, an LA matrix that is a Protein G matrix, a Protein A/G matrix or a Protein L matrix can be used to purify antibodies, antibody fragments comprising an Fc region and Fc fusion proteins.

Other non-limiting examples of LA matrices, and the types of proteins that they are effective in purifying include the following: an immobilized metal ion affinity chromatography (IMAC) column (for purification of proteins with an affinity for metal ions, such as histidine-tagged proteins), a calmodulin resin column (for purification of proteins tagged with calmodulin binding peptide (CBP)), a MEP HYPERCEL™ column (a cellulose matrix that selectively binds immunoglobulin), a column that binds maltose binding protein (MBP) (such as a Dextrin SEPHAROSE™ resin that selectively binds proteins tagged with MBP), a column that binds glutathione-S-transferase (GST) (such as a Glutathione SEPHAROSE™ resin that selectively binds proteins tagged with GST) and a column that binds Strep-Tag II (such as a STREP-TACTIN™ Sepharose resin that selectively binds proteins tagged with Strep-Tag II). Furthermore, immunoaffinity matrices, which comprise an antibody as the target affixed to the solid phase, can be used to purify, an antigen of interest that specifically binds to the antibody affixed to the solid phase.

In some embodiments, a solid support is a material presenting a high surface area for binding of a lipoic acid (LA) compound. Such supports will generally have an irregular surface and may for example be porous or particulate, e.g. particles, fibers, webs, sinters or sieves. Particulate materials e.g. beads are generally preferred due to their greater binding capacity, particularly polymeric beads/particles.

Conveniently, a particulate solid support used according to the invention will comprise spherical beads. The size of the beads is not critical, but they may for example be of the order of diameter of at least 0.01 μm, and have a maximum diameter of preferably not more than 10 and more preferably not more than 6 μm. For example, beads of diameter 1.0 μm, 2.8 μm and 4.5 μm have been shown to work well.

Monodisperse particles, that is those which are substantially uniform in size (e.g. size having a diameter standard deviation of less than 5%) have the advantage that they provide very uniform reproducibility of reaction. Monodisperse polymer particles produced by the technique described in U.S. Pat. No. 4,336,173 are especially suitable.

The particles can be composed of the same polymer throughout, or they can be core-shell polymers as described, for example, in U.S. Pat. No. 4,703,018 and EP-A-0280556 where the shell polymer has the requisite reactive groups.

Non-magnetic polymer beads suitable for use in the method of the invention are available from Dynal Biotech AS (Oslo, Norway) under the trademark DYNOSPHERES, as well as from Qiagen, GE Healthcare Life Sciences, Serotec, Seradyne, Merck, Nippon Paint, Chemagen, Promega, Prolabo, Polysciences, Agowa and Bangs Laboratories.

However, to aid manipulation and separation of immobilized material, and also to facilitate automation if required, magnetizable (“magnetic”) beads are preferred. The term “magnetic” as used herein means that the support is capable of having a magnetic moment imparted to it when placed in a magnetic field, and thus is displaceable under the action of that field. In other words, a support comprising magnetic particles may readily be removed from other components of a sample by magnetic aggregation, which provides a quick, simple and efficient way of separating the particles following the binding of biotin-binding protein, such as a rhizavidin protein as described herein. In addition, such magnetic aggregation is a far less rigorous method of separation than traditional techniques such as centrifugation which generate shear forces which may disrupt cells or degrade any other moieties, e.g. proteins or nucleic acids attached to the biotin-binding protein, e.g., rhizavidin protein.

Thus, the magnetic particles with a biotin-binding protein, e.g., rhizavidin protein attached via conjugation to a lipoic acid (LA) compound, may be removed onto a suitable surface by application of a magnetic field, e.g. using a permanent magnet. It is usually sufficient to apply a magnet to the side of the vessel containing the sample mixture to aggregate the particles to the wall of the vessel and to remove the remainder of the sample so that the remaining sample and/or the particles are available for any desired further steps.

Alternatively, the method for isolating and purifying the rhizavidin and rhizavidin complexes and fusion proteins thereof may be performed using an automated system for handling of such magnetic particles. For example, a sample containing a rhizavidin protein or rhizavidin complexes and fusion proteins thereof may be transferred to such an apparatus, and magnetic particles carrying lipoic acid compound, can be added. The isolated support-bound rhizavidin proteins or fusion proteins or complexes thereof may be washed if desired, and transferred to other vials containing the displacement lipoic acid, followed by removal of the released particles. Particular mention may be made in this regard of the Bead Retriever, available from Dynal Biotech AS, Norway. The apparatus has a system for ready and efficient transfer of the support (carrying a rhizavidin protein or rhizavidin complexes and fusion proteins thereof) from one well to another.

Preferably such magnetic particles are superparamagnetic to avoid magnetic remanence and hence clumping, and advantageously are monodisperse (i.e. are substantially uniform in size, e.g. size having a diameter standard deviation of less than 5%) to provide uniform kinetics and separation. The preparation of superparamagnetic monodisperse particles is described by Sintef in EP-A-106873.

The well-known monodisperse polymeric superparamagnetic beads sold by Dynal Biotech AS (Oslo, Norway) under the trade mark DYNABEADS, are exemplary of commercially available magnetic particles which may be used or modified for use according to the invention.

The solid support of the matrix according to the invention can be of any suitable well-known kind. A conventional affinity separation matrix is often of organic nature and based on polymers that expose a hydrophilic surface to the aqueous media used, i.e. expose hydroxy (—OH), carboxy (—COOH), carboxamido (—CONH₂, possibly in N— substituted forms), amino (—NH₂, possibly in substituted form), oligo- or polyethylenoxy groups on their external and, if present, also on internal surfaces. In one embodiment, the polymers may, for instance, be based on polysaccharides, such as dextran, starch, cellulose, pullulan, agar, agarose etc, which advantageously have been cross-linked, for instance with bisepoxides, epihalohydrins, 1,2,3-trihalo substituted lower hydrocarbons, to provide a suitable porosity and rigidity. In the most preferred embodiment, the solid support is porous agarose beads. The supports used in the present invention can easily be prepared according to standard methods, such as inverse suspension gelation. Alternatively, the base matrices are commercially available products, such as SEPHAROSE™ FF (GE Healthcare). In an embodiment, which is especially advantageous for large-scale separations, the support has been adapted to increase its rigidity, and hence renders the matrix more suitable for high flow rates.

Alternatively, the solid support is based on synthetic polymers, such as polyvinyl alcohol, polyhydroxyalkyl acrylates, polyhydroxyalkyl methacrylates, polyacrylamides, polymethacrylamides etc. In case of hydrophobic polymers, such as matrices based on divinyl and monovinyl-substituted benzenes, the surface of the matrix is often hydrophilised to expose hydrophilic groups as defined above to a surrounding aqueous liquid. Such polymers are easily produced according to standard methods, see e.g. “Styrene based polymer supports developed by suspension polymerization” (R Arshady: Chimica e L'Industria 70(9), 70-75 (1988)). Alternatively, a commercially available product, such as SOURCE™ (GE Healthcare) is used.

In another alternative, the solid support according to the invention comprises a support of inorganic nature, e.g. silica, zirconium oxide etc. In yet another embodiment, the solid support is in another form such as a surface, a chip, capillaries, or a filter. As regards the shape of the LA matrix according to the invention, in one embodiment the LA matrix is in the form of a porous monolith. In an alternative embodiment, the LA matrix is in beaded or particle form that can be porous or non-porous. Matrices in beaded or particle form can be used as a packed bed or in a suspended form. Suspended forms include those known as expanded beds and pure suspensions, in which the particles or beads are free to move. In case of monoliths, packed bed and expanded beds, the separation procedure commonly follows conventional chromatography with a concentration gradient. In case of pure suspension, batch-wise mode will be used.

The lipoic acid compound can be attached to the support via conventional coupling techniques utilising, e.g. amino and/or carboxy groups present in the ligand. Bisepoxides, epichlorohydrin, CNBr, N-hydroxysuccinimide (NHS) etc are well-known coupling reagents. In some embodiments, between the support and the lipoic acid compound, a molecule known as a spacer can be introduced, which improves the availability of the lipoic acid compound and facilitates the chemical coupling of the lipoic acid compound to the support. Alternatively, the lipoic acid compound may be attached to the support by non-covalent bonding, such as physical adsorption or biospecific adsorption. The lipoic acid compound content of the matrix may e.g. be 5-15 mg/ml matrix and can advantageously be 5-10 mg/ml.

In some embodiments, a lipoic acid compound can be coupled to the support by thioether bonds. Methods for performing such coupling are well-known in this field and easily performed by the skilled person in this field using standard techniques and equipment.

A lipoic acid (LA) compound may be covalently attached to a suitable support through reactive groups on the substrate surface by methods well known in the art. These include, for example, attachment through hydroxyl, carboxyl, aldehyde or amino groups which may be provided by treating the immobilizing support to provide suitable surface coating.

Alternatively, supports with functionalized surfaces are commercially available from many manufacturers, such as those particle manufacturers described above. Magnetic particles with the following functionalized surfaces are available from Dynal Biotech AS, Oslo, Norway, for example:

Hydrophobic Beads including, but not limited to: DYNABEADS® M-450 Epoxy (with epoxy groups); DYNABEADS® M-450 Tosylactivated (with tosyl groups); DYNABEADS® M-280 Tosylactivated (with tosyl groups); DYNABEADS® MyOne™ Tosylactivated (with tosyl groups); DYNABEADS® M-500 Subcellular (with tosyl groups).

Hydrophilic Beads, including but not limited to: DYNABEADS® M-270 Epoxy (with epoxy groups); DYNABEADS® M-270 Carboxylic acid (with carboxylic acid groups); DYNABEADS® MyOne Carboxylic acid (with carboxylic acid groups) and Dynabeads M-270 Amine (with amino groups).

The appropriate choice of surface to attach a lipoic acid (LA) compound may depend on the type of biotin-binding protein to be captured, e.g., or moieties which are attached to the biotin-binding protein (such as Rhizavidin protein),—including if it is a fusion protein, or if the biotin-binding protein (e.g., Rhizavidin protein exists as a complex with other proteins or molecules).

Reagents suitable for cross-linking of the solid surface and a lipoic acid (LA) compound include cyanogen bromide, carbonyldiimidazole, glutaraldehyde, hydroxysuccinimide and tosyl chloride. Both Tosyl- and epoxy surfaces have been found to work well with the present invention.

Without wishing to be bound by theory, a lipoic acid (LA) compound can be immobilized, e.g., onto a bead, a magnetic material, a column, a gel and the like. The bead can be magnetized. See, e.g., the U.S. Patents noted above for making and using magnetic particles in purification techniques, and, describing various biotin-avidin binding systems and methods for making and using them, U.S. Pat. Nos. 6,287,792; 6,277,609; 6,214,974; 6,022,688; 5,484,701; 5,432,067; 5,374,516.

### Biotin-Binding Domains and Rhizavidin Proteins

In some embodiments, the methods, compositions and kits can be used to isolate and purify any biotin-binding domain, including “biotin-binding proteins” that comprise such a biotin-binding domain. In some embodiments, a biotin-binding protein which is a monomeric streptavidin or streptavidin-like molecule, such as, for example rhizavidin from Rhizobium etli, as disclosed herein.

Unlike other avidin proteins, such as strepavidin which have a tetrametic quaternary structure, Rhizavidin is unique in that it is dimeric. The tetrameric strepavidin protein has strong binding affinity for biotin and results in a conformational change when ligand binding (e.g., biotin binding) occurs. In contrast, the dimeric rhizavidin protein has a more closed protein confirmation that does not undergo a conformational change when ligand (e.g., biotin) binding occurs.

The full length Rhizavidin protein was first is described Helppolainen et al. (Biochem J., 2007, 405: 397-405), where they describe a 24-amino acid signal sequence at the N-terminus. Helppolainen report that removing the first 24 residues (amino acids 1-24) of the full length Rhizavidin of SEQ ID NO: 4. The inventors have previously demonstrate in US application US2014/0154287 (which is incorporated herein in its entirety by reference), that the first 44 residues of full length Rhizavidin (i.e., amino acids 1-44 of SEQ ID NO: 4) are not necessary for the core structure and biological function of Rhizavidin to bind to biotin. The inventors also previously demonstrated that amino acids 25-44 of SEQ ID NO: 4 (i.e., amino acids MIRTNAVAALVFAVATSALA, SEQ ID NO: 2) of the full length Rhizavidin reduce the solubility and secretion of Rhizavidin expressed in E. coli, as increased solubility and increased secretion was detected when they replaced the first 44 residues of full length Rhizavidin with a different signal peptide, e.g., an E. coli signal peptide.

In some embodiments, a biotin-binding protein is a fragment of wild-type Rhizavidin protein that lacks amino acids 1-44 of SEQ ID NO: 4, i.e., lacks amino acids MIIT SLYATFGTIADGRRTS GGKTMIRTNAVAALVF AVAT S ALA (SEQ ID NO: 5) of the wild-type of rhizavidin of SEQ ID NO: 4. In some embodiments, a biotin-binding protein is a fragment of Rhizavidin that comprises at least 80 consecutive amino acids of the amino acid sequence of:

(SEQ ID NO: 1)
FDASNFKDFSSIASASSSWQNQSGSTMIIQVDSFGNVSGQYVNRAQGTG
CQNSPYPLTGRVNGTFIAFSVGWNNSTENCNSATGWTGYAQVNGNNTEI
VTSWNLAYEGGSGPAIEQGQDTFQYVPTTE NKSLLKD.

In some embodiments, the biotin-binding domain comprises an amino acid sequence having at least 50% amino acid identity, at least 55% identity, at least 60% identity, at least 65% identity, at least 70% identity, at least 75% identity, at least 80% identity, preferably at least 85% identity, at least 90% identity, at least 95% amino acid identity, at least 96% amino acid identity, at least 97% amino acid identity, at least 98% amino acid identity, or at least 99% amino acid sequence identity to SEQ ID NO: 1. In some embodiments, the biotin-binding domain comprises an amino acid sequence having at least 70% identity, at least 75% identity, at least 80% identity, preferably at least 85% identity, at least 90% identity, at least 95% amino acid identity, at least 96% amino acid identity, at least 97% amino acid identity, at least 98% amino acid identity, or at least 99% amino acid sequence identity to SEQ ID NO: 1, and having at least a 80% of the biological activity of SEQ ID NO: 1 with respect to binding to biotin.

In some embodiments, the biotin-binding protein described herein, the biotin-binding domain can be extended on the N- or C-terminus by one or more amino acids with the proviso that the N-terminus of the biotin-binding domain does not comprise an amino acid sequence corresponding to an amino acid sequence 1-44 of the wild-type Rhizavidin. As disclosed herein, the inventors have previously discovered that truncating the first 44 amino acids on the N-terminus of the wild type Rhizavidin can dramatically increase expression of the biotin-binding protein in soluble form in E. coli. Thus, the biotin-binding protein described herein can comprise the sequence X¹-X²-X³, wherein X² is a peptide having the amino acid sequence corresponding to amino acids 45-179 of the wild-type Rhizavidin of SEQ ID NO:4 and X¹ and X³ are independently absent, or X¹ is absent and X³ is present, or vice versa, X¹ is present and X³ is absent, and where X¹ and X³ are present, they can be a peptide of 1 to about 1000 amino acids with the proviso that the N-terminus of X¹ does not comprise an amino acid sequence corresponding to N-terminus of amino acids 1-44 of the wild-type Rhizavidin of SEQ ID NO:4. In some embodiments, for example, where the biotin-binding protein comprises the sequence X¹-X²-X³, X² is a peptide having the amino acid sequence comprising SEQ ID NO: 1 or a protein of at least 80% sequence identity thereto, and X¹ is a signal peptide (or leader sequence) and X³ comprises one or more proteins, e.g., one or more, e.g., at least 1, or at least 2 or at least 3 or at least 4 or more proteins, such as, for example, protein antigens.

In some embodiments, a biotin-binding protein described herein can be a fusion protein comprising a rhizavidin protein of SEQ ID NO: 1 (e.g., referred to as Rhavi) or a protein of at least 80% sequence identity thereto, where the fusion protein can be, for example, Rhavi-A, A-Rhavi, Rhavi-A-A; Rhavi-A-B; Rhavi-A-C, Rhavi-A-B-C, and the like, where A, B and C are separate proteins, and in some embodiments, are antigens, an can be in some embodiments protein antigens or polysaccharide antigens.

In some embodiments, a biotin-binding protein is a fusion protein comprising a C-terminal of SEQ ID NO: 1 (or a protein of at least 80% or 85% or more sequence identity thereto) fused to at least two protein antigens. In some embodiments, the antigens may be the same antigens (e.g., a fusion protein of SEQ ID NO: 1-A-A), or alternatively different protein antigens (e.g., a fusion protein of SEQ ID NO: 1-A-B), where A and B are different antigens.

In some embodiments, the biotin-binding proteins can comprise a signal peptide conjugated to the N-terminus of the biotin-binding protein, i.e. X¹ can comprise a signal peptide. The signal peptide can also be called a leader peptide at the N-terminus, which may or may not be cleaved off after the translocation through the membrane. Secretion/signal peptides are described in more detail in US2014/0154287, which is incorporated herein in its entirety by reference, and are encompassed for use in the present invention.

In some embodiments, the biotin-binding proteins can comprise a signal peptide conjugated to the N-terminus of the biotin-binding protein, i.e. where X¹ comprises a signal peptide. A signal peptide is also called a leader peptide in the N-terminus, which may or may not be cleaved off after the translocation through the membrane. In some embodiments, the E. coli signal sequence is the Dsba signal sequence which comprises at least MKKIWLALAGLVLAFSASA (SEQ ID NO: 7) or MKKIWLALAGLVLAFSASAAQDP (SEQ ID NO: 8). In some embodiments, the signal sequence is MKKVAAFVALSLLMAGC (SEQ ID NO: 9). Secretion/signal peptides are described in more detail below. In some embodiments, the signal sequence is MKKIWLALAGLVLAFSASA (SEQ ID NO: 10), MAPFEPLASGILLLLWLIAPSRA (SEQ ID NO: 11), MKKVAAFVALSLLMAGC (SEQ ID NO: 6), or a derivative or functional portion thereof. The signal sequence can be fused with the sequence comprising amino acids 45-179 of wild-type rhavi by a flexible peptide linker.

In some embodiments, the biotin-binding protein is a fusion protein comprising rhizavidin and one or more proteins, for example, but not limited to antigens. For example, the C-terminal of SEQ ID NO: 1 (or a protein of at least 80% or 85% or more sequence identity thereto) is fused to at least 1, or at least 2 or at least 3, or at least 4 or more proteins, e.g., antigens.

In some embodiments, the methods, compositions and kits as disclosed herein can be used to purify a fusion protein comprising biotin-binding protein and an antigen. In some embodiments, the methods, compositions and kits as disclosed herein can be used to purify a protein comprising biotin-binding protein associated with, or complexed (e.g., with a covalent bond, e.g., by cross-linking, or alternatively via a non-covalent bond) and an antigen.

In some embodiments, the methods, compositions and kits as disclosed herein can be used to purify a fusion protein comprising an antigen (e.g., a protein or peptide antigen) and a rhizavidin protein of that comprises at least 80 consecutive amino acids of the amino acid sequence of SEQ ID NO: 1, or where the fusion protein comprises an amino acid sequence having at least 50% amino acid identity, at least 55% identity, at least 60% identity, at least 65% identity, at least 70% identity, at least 75% identity, at least 80% identity, preferably at least 85% identity, at least 90% identity, at least 95% amino acid identity, at least 96% amino acid identity, at least 97% amino acid identity, at least 98% amino acid identity, or at least 99% amino acid sequence identity to SEQ ID NO: 1. In some embodiments, the fusion protein comprises a rhizavidin protein and an antigen, e.g., a pneumococcal antigen, for example, such as, but not limited to, a pneumococcal proteins of SP0010, SP0043, SP0079, SP0084, SP0092, SP0098, SP0106, SP0107, SP0127, SP0149, SP0191, SP0198, SP0249, SP0321, SP0346, SP0402, SP0453, SP0564, SP0582, SP0589, SP0601, SP0604, SP0617, SP0620, SP0629, SP0648, SP0659, SP0662, SP0664, SP0678, SP0724, SP0742, SP0757, SP0785, SP0787, SP0872, SP0878, SP0899, SP1002, SP1026, SP1032, SP1069, SP1154, SP1267, SP1376, SP1386, SP1404, SP1405, SP1419, SP1479, SP1500, SP1545, SP1560, SP1624, SP1652, SP1683, SP1826, SP1872, SP1891, SP1897, SP1942, SP1966, SP1967, SP1998, SP2048, SP2050, SP2083, SP2084, SP2088, SP2145, SP2151, SP2187, SP2192, SP2197, SP2207 and SP2218, or fragments thereof, which are disclosed in US2015/0374811, which is incorporated herein in its entirety by reference.

In some embodiments, the fusion protein comprises a rhizavidin protein and an antigen, for example, a pathogenic antigen, or a cancer antigen or other antigen. Pathogenic antigens are well known in the art, for example, are disclosed in US patent Application 2014/0154287 or U.S. provisional application 62/477,618 filed on Mar. 28, 2017, which are incorporated herein in their entirety by reference, as are cancer antigens. Exemplary pathogenic antigens and cancer antigens are disclosed herein. In some embodiments, the methods and compositions and kits as disclosed herein can be used for isolation of a rhizavidin protein as disclosed in US patent Application 2014/054286 and U.S. provisional application 62/477,618 filed on Mar. 28, 2017, which are incorporated herein in their entirety by reference.

In some embodiments, an antigen fused or complexed with a biotin-binding protein, e.g., a rhizavidin protein of SEQ ID NO: 1 is any antigen associated with a pathology, for example an infectious disease or pathogen, or cancer or an immune disease such as an autoimmune disease. In some embodiments, an antigen can be expressed by any of a variety of infectious agents, including virus, bacterium, fungus or parasite. An antigen can also include, for example, pathogenic peptides, toxins, toxoids, subunits thereof, or combinations thereof (e.g., cholera toxin, tetanus toxoid). Exemplary antigens include, for example, but are not limited to, pneumococcal antigens, tuberculosis antigens, anthrax antigens, HIV antigens, Acinetobacter antigens, Clostridium difficile antigens, enteric Gram-negative bacterial antigens (e.g., E. coli antigens, Salmonella antigens, Enterobacter antigens, Klebsiella antigens, Citrobacter antigens, Serratia antigens), non-enteric Gram-negative bacterial antigens (e.g., Pertussis antigens, Meningococcal antigens, Haemophilus antigens, Pseudomonas antigens), Gram-positive bacterial antigens, seasonal or epidemic influenza antigens, Staphylococcus aureus antigens, Haemophilus antigens, HPV antigens, toxoids, toxins or toxin portions, fungal antigens, viral antigens, cancer antigens, or combinations thereof.

In some embodiments, other biotin-binding proteins can be isolated using the methods as disclosed herein. Such biotin-binding proteins include, but are not limited to a polypeptide that is able to bind lipoic acid or to bind a compound comprising a lipoic acid compound as disclosed herein with a dissociation constant of equal or less than 10⁻⁷ mol/l, 10⁻⁸ mol/l, 10⁻⁹ mol/l, 10⁻¹⁰ mol/l or 10⁻¹¹ mol/l. Examples for such biotin-binding proteins include, without being restricted to, streptavidin (UniProt P22629), avidin (UniProt P02701), rhizavidin (UniProt Q8KKW2) or variants and functional homologues thereof.

In some embodiments, a biotin-binding protein is, but is not limited to a polypeptide that is able a biotin or to bind a compound comprising a biotin moiety with a dissociation constant of equal or less than 10-7 mol/l, 10⁻⁸ mol/l, 10⁻⁹ mol/l, 10⁻¹⁰ mol/l or 10⁻¹¹ mol/l. Examples for such biotin-binding proteins include, without being restricted to, streptavidin (UniProt P22629), avidin (UniProt P02701), rhizavidin (UniProt Q8KKW2) or variants and functional homologues thereof. UniProt numbers contained in the present specification refer to entries in the Protein knowledgebase (Swiss Institute of Bioinformatics).

In some embodiments, a rhizavidin protein, or fusion protein thereof for isolation and purification according to the methods as disclosed herein can comprise a lipidation sequence at the N-terminus, e.g., MKKVAAFVALSLLMAGC (SEQ ID NO: 6) or a an amino acid 85% identity thereto.

In some embodiments, a rhizavidin protein or fusion protein thereof can comprise a signal peptide linked to the N-terminus of the biotin-binding domain either directly (e.g., via a bond) or indirectly (e.g., by a linker). In some embodiments, the signal peptide can be linked to the N-terminus of the biotin-binding domain by a peptide linker. The peptide linker sequence can be of any length. For example, the peptide linker sequence can be one, two, three, four, five, six, seven, eight, nine, ten, eleven, twelve, thirteen, fourteen, fifteen or more amino acids in length. In some embodiments, the peptide linker is four amino acids in length.

The peptide linker sequence can comprise any amino acid sequence. For example, the peptide linker can comprise an amino acid sequence which can be cleaved by a signal peptidase. In some embodiments, the peptide linker comprises the amino acid sequence AQDP (SEQ ID NO: 12) or VSDP (SEQ ID NO: 13).

In some embodiments, a rhizavidin protein, or fusion protein thereof for isolation and purification according to the methods as disclosed herein can be conjugated at its C-terminus to a peptide of 1-100 amino acids. Such peptides at the C-terminus can be used for, example, but not limited to: purification tags, linkers to other domains, and the like. In some embodiments, a rhizavidin protein, or fusion protein thereof for isolation and purification according to the methods as disclosed herein comprises on its N- or C-terminus one or more (e.g., one, two, three, four, five, six, seven, eight, nine, ten or more) purification tags. Examples of purification tags include, but are not limited to a histidine tag, a c-my tag, a Halo tag, a Flag tag, and the like. In some embodiments, the biotin-binding protein comprises on its C-terminus a histidine tag, e.g. a (His)₆ (SEQ ID NO. 14). In some embodiments, a rhizavidin protein, or fusion protein thereof for isolation and purification according to the methods as disclosed herein comprises a peptide of amino acid sequence GGGGSSSVDKLAAALEHHHHHH (SEQ ID NO: 15). This peptide at the C-terminus provides a histidine tag for purification and a place for insertion of other domains, e.g. antigenic domains, in the biotin protein. Further, while Helppolainen et al. (Biochem J., 2007, 405: 397-405) describe expression of Rhizavidin in E. coli, there is no teaching or suggestion in Helppolainen et al. for conjugating an additional peptide to the C-terminus of the biotin-binding domain of Rhizavidin.

A purification tag can be conjugated to a rhizavidin protein, or fusion protein thereof for isolation and purification according to the methods as disclosed herein by a peptide linker to enhance the probability that the tag is exposed to the outside. The length of the linker can be at least one (e.g., one, two, three, four, five six, seven, eight, nine, ten, eleven, twelve, thirteen, fourteen, or fifteen) amino acid. The linker peptide can comprise any amino acid sequence without limitations. In some embodiments, the linker peptide comprises the amino acid sequence VDKLAAALE (SEQ ID NO: 16) or GGGGSSSVDKLAAALE (SEQ ID NO: 17). In some embodiments, a rhizavidin protein, or fusion protein thereof for isolation and purification according to the methods as disclosed herein can comprise at its C-terminus the amino acid sequence VDKLAAALEHHHHH (SEQ ID NO: 18) or GGGGSSSVDKLAAALEHHHHHH (SEQ ID NO: 19). Other purification tags are known and are encompassed for use herein. In some embodiments, the purification tags can be cleaved or removed after the rhizavidin protein or fusion protein thereof are purified. In some embodiments, the biotin-binding protein, e.g., rhizavidin protein or fusion protein thereof does not comprise a purification tag.

In some embodiments, a rhizavidin protein, or fusion protein thereof for isolation and purification according to the methods as disclosed herein has a spacer peptide, e.g., a 14-residue spacer (GSPGISGGGGGILE) (SEQ ID NO: 20) separating a protein, e.g., an antigen from the rhizavidin protein. The coding sequence of such a short spacer can be constructed by annealing a complementary pair of primers. One of skill in the art can design and synthesize oligonucleotides that will code for the selected spacer. Spacer peptides should generally have non-polar amino acid residues, such as glycine and proline.

### Lipidated Rhizavidin Fusion Protein or Biotin-Binding Protein

In another aspect provided herein is the purification and isolation of a lipidated biotin-binding protein, e.g., a lipidated rhizavidin protein or fusion protein thereof. As used herein, the term “lipidated biotin-binding protein” refers to a biotin-binding protein that is covalently conjugated with a lipid. The lipid moieties could be a diacyl or triacyl lipid.

In some embodiments, a rhizavidin protein, or fusion protein thereof for isolation and purification according to the methods as disclosed herein comprises a lipidation sequence. As used herein, the term “lipidation sequence” refers to an amino acid sequence that facilitates lipidation in a bacteria, e.g., E. coli, of a polypeptide carrying the lipidating sequence. The lipidation sequence can be present at the N-terminus or the C-terminus of the protein. The lipidation sequence can be linked to the recombinant biotin-binding protein to form a fusion protein, which is in lipidated form when expressed in E. coli by conventional recombinant technology. In some embodiments, a lipidation sequence is located at the N-terminus of the biotin-binding protein.

Any lipidation sequence known to one of ordinary skill in the art can be used. In some embodiments, the lipidating sequence is MKKVAAFVALSLLMAGC (SEQ ID NO: 9) or a derivative or functional portion thereof. Other exemplary liquidating sequences include, but are not limited to, MNSKKLCCICVLFSLLAGCAS (SEQ ID NO: 21), MRYSKLTMLIPCALLLSAC (SEQ ID NO: 22), MFVTSKKMTAAVLAITLAMSLSAC (SEQ ID NO: 23), MIKRVLVVSMVGLSLVGC (SEQ ID NO: 24), and derivatives or functional portions thereof.

In some embodiments, the lipidation sequence can be fused to a rhizavidin protein or fusion protein thereof via a peptide linker, wherein the peptide linker attaches the lipidating sequence to the biotin-binding protein. In some embodiment, the peptide linker comprises the amino acid sequence VSDP (SEQ ID NO: 25) or AQDP (SEQ ID NO: 26).

In some embodiments, a rhizavidin protein, or fusion protein thereof for isolation and purification according to the methods as disclosed herein that is a lipoprotein as described herein have enhanced immunogenicity. Without wishing to be bound by a theory, lipid moieties at the N-terminals of the lipoproteins or lipopeptides contribute to the adjuvant activity.

In some embodiment, a biotin-binding protein isolated and purified using the methods, compositions and kits as disclosed herein is a streptavidin-rhizavidin chimera, which has high affinity for biotin but can be expressed as a monomer (See Lim, K H, Huang, H, Pralle, A, Park, S. Stable, High-Affinity Streptavidin Monomer for Protein Labeling and Monovalent Biotin Detection. Biotechnol Bioeng. 2013 January; 1 10(1):57-67 (which may be found at world-wide web at “ncbi. nlm.nih.qov/pubmed/228G8584”), or as disclosed in WO2015/095603, which is incorporated herein in its entirety by reference.

In some embodiment, a biotin-binding protein isolated and purified using the methods, compositions and kits as disclosed herein is avidin, streptavidin, or bradavidin (Brad), neutravidin, AVR protein, Tamavidin 1, Tamavidin 2 or homologues, fusion proteins, chimeras or variants thereof.

Biotin-binding proteins isolated and purified using the methods, compositions and kits as disclosed herein can be, for example. avidin, streptavidin, neutravidin, AVR protein (Biochem J, (2002), 363: . . . 609-617), Buradabijin (Bradavidin) (J Biol Chem, and (2005), 280: 13250-13255), Rizabijin (Rhizavidin) (Biochem J., (2007.), 405: 397-405), tamavidin 1 or tamavidin 2 (as disclosed in WO02/072817, US20120064543A1 which are incorporated herein in in their entirety by reference), and their variants such as that bind strongly to biotin. Preferably, at least the dissociation constant of biotin (KD) of 10 ⁻⁶, more preferably 10 ⁻⁸ or less, more preferably 10 ⁻¹⁰ or less.

In some embodiments, tamavidin and its variants highly expressed in E. coli. Tamavidin is a biotin-binding protein that has been found from Basidiomycetes cornucopiae (Pleurotus conucopiae) is an edible mushroom (WO02/072817, Takakura et al (2009) FEBS J 276: 1383-1397). As a variant of tamavidin, for example, like a high binding capacity and low non-specific binding tamavidin (PCT/JP2009/64302). The sequences of tamavidin 1 and tamavidin 2 and variants thereof are disclosed in US20120064543A1, which is incorporated herein in its entirety by reference. Tamavidin 2 is a fungal avidin-like protein that binds biotin with high affinity and is highly produced in soluble form in Escherichia coli (see Takakura et al., J Biotechnol. 2010, 15;145(4):317-22.)

In some embodiments, a biotin-binding protein isolated and purified using the methods, compositions and kits as disclosed herein is a monomeric streptavidin, or a streptavidin variant, as disclosed in Dubdas et al., “Streptavidin-binding technology; improvements and innovations in chemical and biological applications”, Applied Microbiol Biotech, 2013; DOI; 10.1007/s00253-013-5232-z, which is incorporated herein in its entirety by reference.

### Methods for Preparing LA-Matrix

The general procedure for preparing the lipoic acid matrix or resin useful in the methods and compositions as disclosed herein includes covalently attaching a lipoic acid (LA) compound to the particles using generally known reactions. Details of a representative procedure are illustrated in EXAMPLE 1 below, entitled “Preparation of the Lipoic acid resin (LA-matrix)”.

One embodiment of the present invention provides a process for the recovery of a rhizavidin protein, or a complex, or fusion protein comprising the same, in a method employing a lipoic acid (LA) compound immobilized to a solid surface, where in some embodiments, the method comprises the following steps:

(i) Preparing a lipoic acid compound matrix (LA-matrix) as disclosed herein in the Examples.

(ii) Purifying a biotin-binding protein, e.g., rhizavidin or a complex or rhizavidin fusion protein comprising a rhizavidin protein from a biological sample. In some embodiments, the biological sample is a bacterial culture sample, or any biological sample obtained from expressing a rhizavidin, or rhizavidin fusion protein, e.g., large or small scale recombinant protein expression systems including, but not limited to, in vitro (cell-free) protein expression systems, bacterial protein expression systems, viral expression systems such as bacliovirus expression systems, insect expression systems, eukaryotic expression systems, mammalian expression systems and chemical protein synthesis, all of which are well known in the art.

For simplicity, and without wishing to be bound by theory, applicants herein describe the steps for purification of rhizavidin or rhizavidin fusion protein expressed using a bacterial expression system using a LA-matrix as described herein;
i. After bacterial expression of the rhizavidin or rhizavidin fusion protein, the bacterial suspension is centrifuged and the pellet collected and resuspended in an appropriate lysis buffer, such as, for example, 20 mM Tris, 1M NaCl, pH8.0. Any standard bacterial lysis buffer is encompassed for use, for example, a lysis buffer comprising between 0.75M and 1.5M NaCl and having a pH of between 7.5 and 9.0. In some embodiments, a lysis buffer can optionally comprise DNase, RNase and proteinase inhibitors and optionally 10 mM MgCl₂. Lysis of the bacterial cells can optionally be aided by sonication or other such approaches, after which the cell lysate is collected by centrifugation.
ii. The cell lysate from step (i) is added to a LA matrix as disclosed herein, and mixed well and incubated for an appropriate amount of time to allow the rhizavidin or rhizavidin fusion protein to bind to the lipoic acid compound. Typically, incubation times can be, for example, at least 1 hour, or at least 2 hours, or at least about 3 hr, or at least about 4 hr, or at least about 5 hr, or at least about 6 hr, or at least about 12 hr, or at least about 24 hr, or more than 24 hours, but less than 3-days. In some embodiments, incubation is for about 2-4 hrs at 4° C., while shaking. In some embodiments, before the cell lysate is added to the LA matrix, the LA matrix is equilibrated with the lysis buffer used to lyse the cells, or other buffer solution which comprises the rhizavidin or rhizavidin fusion protein to be isolated.
iii. After incubation of the LA matrix with the sample comprising the rhizavidin or rhizavidin fusion protein, the LA matrix is washed with an appropriate with wash buffer, for example, but not limited to, 50% ethanol, 20 mM Tris, 1M NaCl. Any wash buffer known by an ordinary skilled artisan is encompassed for use as a wash buffer herein. Washing is stopped when no protein is detected in flow through (elutant).
iv. Release of the rhizavidin or rhizavidin fusion from the LA matrix occurs by eluting the protein with elution buffer, e.g., a solution comprising excess lipoic acid at pH of about 7.5-8.5. In some embodiments, a suitable elutant buffer comprises Lipoic acid (at about 1-10 mg/ml, e.g., about 2.5 mg/ml of Lipoic acid) in 20 mM Tris, 1M NaCl, 5% ethanol at pH8.

A person of ordinary skill in the art can scale up the volume of the lipoic acid matrix as disclosed herein. For example, a LA matrix having a volume of 10 ml, or about 20 ml, or about 30 ml, or about 40 ml, or about 50 ml, or about 60 ml, or about 70 ml, or about 80 ml, or about 90 ml, or about 100 ml, or between 100-150 ml, or between 150-200 ml, or between 200-300 ml, or between 300-400 ml or between 400-500 ml or greater than 500 ml can be prepared by one of ordinary skill in the art. In some embodiments, the volumes of the wash buffer, and elution buffers are scaled up accordingly, as is the volume of lysis buffer comprising the biotin-binding protein of interest (e.g., rhizavidin protein, or rhizavidin fusion protein or complex thereof) applied to the LA matrix is scaled up accordingly.

Binding of a rhizavidin protein, or rhizavidin fusion protein or complex thereof to the LA matrix typically is achieved by column chromatography. That is, the LA matrix is formed into a column, a biochemical mixture containing a rhizavidin protein of interest (e.g., a rhizavidin protein, or fusion protein or complex thereof) is flowed through the column, followed by washing of the column by flowing through the column one or more wash solutions, followed by elution of the rhizavidin protein of interest (e.g., a rhizavidin protein, or fusion protein or complex thereof) from the column by flowing through the column an elution buffer.

Alternatively, binding of a rhizavidin protein of interest (e.g., a rhizavidin protein, or fusion protein or complex thereof) to the LA matrix can be achieved by batch treatment, in which the biochemical mixtures containing a rhizavidin protein of interest (e.g., a rhizavidin protein, or fusion protein or complex thereof) is incubated with the LA matrix in a vessel to allow for binding of the rhizavidin protein of interest (e.g., a rhizavidin protein, or fusion protein or complex thereof) to the LA matrix, the solid phase medium is removed from the vessel (e.g., by centrifugation), the solid phase medium is washed to remove impurities and again recovered (e.g., by centrifugation) and the rhizavidin protein of interest (e.g., a rhizavidin protein, or fusion protein or complex thereof) is eluted from the solid phase medium.

In yet another embodiment, a combination of batch treatment and column chromatography can be used. For example, the initial binding of the rhizavidin protein of interest (e.g., a rhizavidin protein, or fusion protein or complex thereof) to the LA matrix can be achieved by batch treatment and then the solid phase medium can be packed into a column, following by washing of the column and elution of the rhizavidin protein of interest (e.g., a rhizavidin protein, or fusion protein or complex thereof) from the column.

The nature of a particular solid phase matrix, in particular the binding properties of the target attached to the solid phase, determines the type(s) of rhizavidin protein of interest (e.g., a rhizavidin protein, or fusion protein or complex thereof) that can be purified using that solid phase matrix. For example, in some embodiments, the LA matrix is a Protein A column, which comprises as the lipoic acid compound attached to the solid phase a bacterial cell wall protein, Protein A. The binding properties of Protein A are well established in the art.

Although the purification of a rhizavidin protein, i.e., a rhizavidin protein of SEQ ID NO: 1 is shown as an exemplary biotin-binding domain purified using the LA affinity chromatography as disclosed herein, it will be readily apparent to the ordinarily skilled artisan that other biotin-binding domains can also be purified using similar methods using the LA matrix as described herein. It will also be readily apparent to the ordinarily skilled artisan that additional steps are carried out both before and after the elution of the biotin-binding protein from the LA-matrix to achieve purification of the biotin-binding protein of interest from the LA affinity chromatography matrix. For example, prior to the washing step, the methods of the invention can include an equilibration step, in which the affinity chromatography LA matrix is equilibrated with a loading buffer (or lysis buffer), and a loading or capture step, in which a biochemical mixture (e.g. cellular harvest) containing the protein of interest is applied to the LA matrix. Suitable conditions for the equilibration and loading buffers will vary depending upon the nature of the LA matrix and the biotin-binding protein of interest to be purified, and the ordinarily skilled artisan can readily determine such conditions using methods and information well established in the art. Non-limiting examples of equilibration and loading (i.e., lysis) buffers for the purification of a rhizavidin protein on a LA matrix column are set forth in Example 1.

Additionally, after the washing step(s) as mentioned above, the methods of the invention can include one or more additional washings step(s) utilizing common wash solutions, and/or an elution step, in which an elution buffer is applied to the affinity chromatography LA matrix to elute the biotin-binding protein of interest from the LA matrix. Suitable conditions for the elation buffer will vary depending upon the nature of the LA matrix and the biotin-binding protein of interest to be purified, and the ordinarily skilled artisan can readily determine such conditions using methods and information well established in the art. Typically, elution of the protein of interest from the LA matrix is carried out at an pH of 8.0-9.5. Non-limiting examples of an elution buffers for the purification of a rhizavidin protein on a LA matrix column is set forth in Example 1.

General methods in molecular and cellular biochemistry can also be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

### Wash Solution

In some embodiments, the washing step in the methods as disclosed herein is effective in removing a variety of impurities, including high molecular weight (HMW) species and host cell proteins (HCPs) that are not the biotin-binding domain or biotin-binding protein.

Efficient removal of impurities, including host cell proteins (HCPs) and product-related impurities such as high molecular weight (HMW) species and low molecular weight (LMW) species, is a crucial factor during downstream processing of the biotin-binding domain, e.g., a rhizavidin protein or fusion protein or complex thereof. In some embodiments, the wash buffer comprises 20 mM Tris, 1M NaCl, pH 8.0.

### Elution Buffer

In another aspect, the invention provides a method of producing a biotin-binding protein, such as, e.g., rhizavidin protein or a fusion protein or complex thereof, using a LA-matrix column, the method comprising (a) loading a mixture comprising the biotin-binding protein, onto the LA-matrix column; (b) washing the LA-matrix column with a wash solution as disclosed herein, wherein the wash solution removes impurities from the LA-matrix column; and (c) eluting the biotin-binding protein, such as, e.g., rhizavidin protein or a fusion protein or complex thereof, from the LA-matrix column.

In some embodiments of the elution step, an eluent (or elution buffer) is passed over the matrix under conditions that provide desorption i.e. release of the biotin-binding domain. Such conditions are commonly provided by a change of the pH, the salt concentration i.e. ionic strength, hydrophobicity etc. Various elution schemes are known, such as gradient elution and step-wise elution. In some embodiments, the elution buffer comprises a competitive substance, which will replace lipoic acid-biotin binding domain interaction. In some embodiments, the competitive substance is, for example, a high concentration of a lipoic acid compound as disclosed herein, or a biotin or biotin derivative as disclosed herein. In alternative embodiments, a competitive substance to release the biotin-binding protein from the LA matrix is biotin, PEG-biotin, iminobiotin, desthiobiotin, diaminobiotin, or variants or derivatives thereof.

In some embodiments, a suitable elution buffer comprises between about 1.0 mg/ml and about 10.0 mg/ml of Lipoic acid in 20 mM Tris, 1M NaCl, 5% ethanol at pH 8.0.

In some embodiments, an elution buffer for use in the methods, kits and compositions as disclosed herein comprises more than 1.0 mg/ml lipoic acid or lipoic acid compound as disclosed herein, and less than 10 mg/ml lipoic acid or lipoic acid compound. In some embodiments, an elution buffer for use in the methods, kits and compositions as disclosed herein comprises about 1.0 mg/ml, or about 1.5 mg/ml, or about 2.0 mg/ml, or about 2.5 mg/ml, or about 3.0 mg/ml, or about 3.5 mg/ml, or about 4.0 mg/ml, or about 4.5 mg/ml, or about 5.0 mg/ml, or about 5.5 mg/ml, or about 6.0 mg/ml, or about 6.5 mg/ml, or about 7.0 mg/ml, or about 7.5 mg/ml, or about 8.0 mg/ml, or about 8.5 mg/ml, or about 9.0 mg/ml, or about 9.5 mg/ml, or about 10.0 mg/ml lipoic acid or lipoic acid compound as disclosed herein.

In some embodiments, an elution buffer for use in the methods as disclosed herein can comprise a nonbuffering salt, which is of a type and at a concentration sufficient to break ionic interactions between impurities and one or more components of the affinity matrix.

As used herein, the term “nonbuffering salt” refers to a salt that is present in the elutant solution that is of a type, and at a concentration, such that it does not substantially contribute to retaining the pH of the wash solution(s) under the applied conditions (such as high pH) upon addition of acid or base. Typically, the nonbuffering salt is an ionic salt. Nonbuffering salts include halogen salts, including those that comprise Cl or Br (more preferably CO, in particular halogen salts comprising alkali metals or alkaline earth metals, including Na, K, Ca and Mg (more preferably Na or K). The term “nonbuffering salt” does not include buffering salts, such as sodium acetate, sodium phosphate and Tris, that do substantially contribute to retaining the pH of a wash solution(s) under the applied conditions. In a preferred embodiment, the nonbuffering salt is a halogen salt (e.g., comprising Cl or Br). In another embodiment, the nonbuffering salt is a halogen salt that comprises sodium (Na), potassium (K), calcium (Ca) or magnesium (Mg), more preferably, sodium (Na) or potassium (K). In yet another embodiment, the nonbuffering salt is selected from the group consisting of NaCl, KCl, CaCl2 and MgCl2. In a particularly preferred embodiment, the nonbuffering salt is sodium chloride (NaCl). Typically, the nonbuffering salt is used at a “high” concentration of at least 1 M. Other suitable concentrations and concentration ranges are described further below.

In some embodiments, the nonbuffering salt is a halogen salt. In a particularly preferred embodiment, the nonbuffering salt is sodium chloride (NaCl). In other embodiments, the nonbuffering salt can be, for example, potassium chloride (KCl), calcium chloride (CaCl2) or magnesium chloride (MgCl2). The concentration of nonbuffering salt in the wash solution typically is between 0.1 M and 2.0 M (e.g., 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M 0.75 M, 0.8 M, 0.85 M, 0.9 M, 0.95 M, 1.0 M, 1.1 M, 1.15 M, 1.20 M, 1.25 M, 1.30 M, 1.35 M, 1.40 M, 1.45 M, 1.5 M, 1.55 M, 1.6 M, 1.65 M, 1.7 M, 1.75 M, 1.8 M, 1.85 M, 1.9 M, 1.95 M, or 2.0 M), or between 0.5 M and 1.5 M (e.g., 0.5 M, 0.55 M, 0.6 M, 0.65 M, 0.7 M, 0.75 M, 0.8 M, 0.85 M, 0.9 M, 0.95 M, 1.0 M, 1.1 M, 1.15 M, 1.2 M, 1.25 M, 1.3 M, 1.35 M, 1.4 M, 1.45 M, or 1.5 M), or between 1 M and 2 M (e.g., 1 M, 1.1 M, 1.15 M, 1.2 M, 1.25 M, 1.3 M, 1.35 M, 1.4 M, 1.45 M, 1.5 M 1.55 M, 1.6 M, 1.65 M, 1.7 M, 1.75 M, 1.8 M, 1.85 M, 1.9 M, 1.95 M, or 2 M). In certain embodiments, the concentration of nonbuffering salt in the elutant solution is 1 M or greater. In particular embodiments, the non-buffering salt in the wash solution is present at a concentration of 0.75 M or about 0.75 M, 1.0 M or about 1.0 M, or 1.25 M or about 1.25 M.

In some embodiment, the pH of the elution buffer solutions as disclosed herein typically is greater than 8.0, although lower pHs are also suitable for use with the elution buffer solution(s) of the invention. In a particular embodiment, the pH is greater than 8.0, preferably at least 8.1, more preferably at least 8.5 or 8.9. In one embodiment, the pH of the one or more wash solutions is in a range of 8.1-9.5. In another embodiment, the pH of the one or more elution buffers is in a range of 8.5-9.5. In another embodiment, the pH of the one or more elution buffers is about 9.0. In another embodiment, the pH of the one or more elution buffers is 9.0. Alternatively, depending on the biotin-binding protein to be purified, a lower pH value can be used, for example a pH in a range of pH 5.0-8.0, or a pH of 7.5 or 7.0 or 6.5 or 5.0. Depending on the properties of the biotin-binding protein to be purified, the ordinarily skilled artisan can select an appropriate pH value for the elution buffer and/or wash solution. Accordingly, the elution buffer solution(s) can contain one or more buffers for adjusting and/or maintaining the pH. Non-limiting examples of typical buffers that can be included in the elution buffer solution(s) include Tris (tris(hydroxymethyl)methylamine), bis-Tris, bis-Tris propane, histidine, triethanolamine, diethanolamine, formate, acetate, MES (2-(N-morpholino)ethanesulfonic acid), phosphate, HEPES (4-2-hydroxyethyl-1-piperazineethanesulfonic acid), citrate, MOPS (3-(N-morpholino)propanesulfonic acid), TAPS (3-{[tris(hydroxy methyl)methyl]amino}propanesulfonic acid), Bicine (N,N-bis(2-hydroxyethyl)glycine), Tricine (N-tris(hydroxymethyl)methylglycine), TES (2-{[tris(hydroxymethyl)methyl]amino}ethanesulfonic acid), PIPES (piperazine-N,N′-bis(2-ethanesulfonic acid), cacodylate (dimethylarsinic acid) and SSC (saline sodium citrate).

In some embodiments, the pH of the one or more elution buffer solutions is greater than 8.0, preferably at least 8.1, more preferably at least 8.5 and even more preferably at least 8.9. In one embodiment, the pH of the one or more elution solutions is in a range of 8.1-9.5. In another embodiment, the pH of the one or more elution solutions is in a range of 8.5-9.5. In another embodiment, the pH of the one or more elution solutions is about 9.0. In another embodiment, the pH of the one or more elution solutions is 9.0.

The environment for the elution buffer can be defined as alkaline, meaning of an increased pH-value, for example above about 8.5, such as up to about 9 or 10.

In some embodiments, the elution step as described herein results in a percent yield of the biotin-binding protein of interest, e.g., a rhizavidin protein or fusion protein or complex thereof, that is greater than 95%, more preferably greater than 96%, even more preferably greater than 97%. With respect to the reduction in HMW species in the eluate, which can be expressed as the % HMW in the eluate, in various embodiments, the elution step as described herein results in a % HMW in the eluate that is less than 10%, or less than 5%, or less than 2.0%, or less than 1% or less than 0.5%. With respect to the reduction in HCPs in the eluate, which can be expressed as the logarithmic reduction value (LRV), in various embodiments, the elution step results in an LRV for HCPs in the eluate that is at least 1.1, or at least 1.3, or at least 1.5, or at least 2.0, or at least 2.3, or at least 2.5, or at least 2.7.

### Uses

For example, this invention may be used in methods for the detection, identification, determination, purification, separation and/or isolation of compounds of biological interest, targets, from heterogeneous mixtures. Such compounds can be defined as a biotin-binding protein, such as Rhizavidin protein as described herein, or a fragment thereof, or any biological or chemical compound which is attached, via covalent attachment (i.e., as a fusion protein or by cross-linkage) or non-covalent attachment (e.g., exists in a complex) to a Rhizavidin protein or fragment as described herein.

The method of the invention may be applied to the purification or isolation of any moiety that is attached to a a biotin-binding protein, such as Rhizavidin protein as described herein, or a fragment thereof which bind to a lipoic acid compound as disclosed herein, e.g., any type of cells or cellular component from any biological sample or artificial media. Representative biological samples derived from a human or animal source include whole blood, and blood-derived products such as plasma, buffy coat or leukophoresis products, serum, saliva, lymph, bile, urine, milk, faeces, cerebrospinal fluid or any other body fluids like spinal fluid, seminal fluid, lacrimal fluid, vaginal secretions, and the like, as well as stool specimens. It is also possible to assay fluid preparations of human or animal tissue such as skeletal muscle, heart, kidney, lungs, brains, bone marrow, skin and the like or cellular extracts or secretions and cell suspensions obtained by density gradient centrifugation etc., and also environmental samples such as soil, water or food samples. Such samples may be used as they are, or they may be subjected to various purification, decontamination, filtration, or concentration methods. The sample may also include relatively pure or partially purified starting materials, such as semi-pure preparations obtained by other cell or biomolecule separation processes like immunomagnetic separation.

Moreover it should be noted that the method according to the invention can be applied to the isolation and subsequent liberation of sub-cellular components such as mitochondria and nuclei, and macromolecules such as proteins and nucleic acids that are attached or associated with the biotin-binding protein, such as Rhizavidin protein as described herein, or a fragment thereof which bind to a lipoic acid compound as disclosed herein. The entity to be isolated may be naturally antigenic or may be made so artificially.

In some embodiments, a moiety associated with the biotin-binding protein, such as Rhizavidin protein as described herein, or a fragment thereof which bind to a lipoic acid compound as disclosed herein can be a particular structural molecule e.g. a peptide, protein, glycoprotein, lipid or carbohydrate etc. associated with the surface of larger biological entities for example cells. Other targets may be biological substances include peptides, polypeptides, proteins, lipoproteins, glycoproteins, nucleic acids (DNA, RNA, PNA, aptamers) and nucleic acid precursors (nucleosides and nucleotides), polysaccharides, lipids such as lipid vesicles. Typical proteins which are detectable in conventional streptavidin/biotin systems, and useful herein, include cytokines, hormones, vitamins surface receptors, haptens, antigens, antibodies, enzymes, growth factors, recombinant proteins, toxins, and fragments and combinations thereof.

The term “cell” is used herein to include all prokaryotic (including archaebacteria and mycoplasma) and eukaryotic cells and other entities such as viruses and sub-cellular components such as organelles (e.g. mitochondria and nuclei). Representative “cells” thus include all types of mammalian and non-mammalian animal cells, plant cells, insect cells, fungal cells, yeast cells, protozoa, bacteria, protoplasts and viruses.

In some embodiments, the methods and compositions and kits as disclosed herein can be used for isolation of complexes or cells associated with a biotin-binding protein, e.g., a rhizavidin protein, or fusion protein thereof, that binds to a lipoic acid compound.

In some embodiments, the methods and compositions and kits as disclosed herein can be used for isolation of a rhizavidin protein as disclosed in US patent Application 2014/054286, which is incorporated herein in its entirety by reference.

In some embodiments, the methods and compositions and kits as disclosed herein can be used for isolation of a biotin-binding protein for use in a multiple antigen presenting (MAPS) complex, disclosed in US patent Application 2014/0154287,which is incorporated herein in its entirety by reference.

In some embodiments, the methods and compositions and kits as disclosed herein can be used for isolation of a plurality of different rhizavidin fusion proteins at the same time, e.g., at least 2, or at least 3, or at least about 4, or between 4-5, or between 5-7, or between 7-10, or between 10-20, or more than 20 but less than 50 different rhizavidin fusion proteins at the same time.

In some embodiments, the methods and compositions and kits as disclosed herein can be used for isolation of a rhizavidin protein comprising at least 70% sequence identity to SEQ ID NO: 1, alone or fused to an antigen, e.g., a pathogenic antigen or cancer antigen or the like. Pathogenic antigens are well known in the art, for example, are disclosed in US patent Application 2014/0154287, as are cancer antigens.

In some embodiments, an antigen fused or complexed with a biotin-binding protein, e.g., a rhizavidin protein of SEQ ID NO: 1 is any antigen associated with a pathology, for example an infectious disease or pathogen, or cancer or an immune disease such as an autoimmune disease. In some embodiments, an antigen can be expressed by any of a variety of infectious agents, including virus, bacterium, fungus or parasite. An antigen can also include, for example, pathogenic peptides, toxins, toxoids, subunits thereof, or combinations thereof (e.g., cholera toxin, tetanus toxoid).

Non-limiting examples of from infectious viruses which antigens can be derived include , e.g., Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), Marek's disease virus, herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions and methods described herein are contemplated for use in treating infections with these viral agents.

Examples of fungal infections from which antigens can be derived include aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans. Components of these organisms can be included as antigens in the MAPS described herein.

Examples an antigen derived from an infectious microbes, include such as Bordatella pertussis, Brucella, Enterococci sp., Neisseria meningitidis, Neisseria gonorrheae, Moraxella, typeable or nontypeable Haemophilus, Pseudomonas, Salmonella, Shigella, Enterobacter, Citrobacter, Klebsiella, E. coli, Helicobacter pylori, Clostridia, Bacteroides, Chlamydiaceae, Vibrio cholera, Mycoplasma, Treponemes, Borelia burgdorferi, Legionella pneumophilia, Mycobacteria sps (such as M. tuberculosis, M. avium, M. intracellulare, M. kansaii, M. gordonae, M. leprae), Staphylococcus aureus, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Leptospira sps., Pasturella multocida, Bacteroides sp., Fusobacterium nucleatum, Streptobacillus moniliformis, Treponema pallidium, Treponema pertenue, and Actinomyces israelli.

Additional parasite pathogens from which antigens can be derived include, for example: Entamoeba histolytica, Plasmodium falciparum, Leishmania sp., Toxoplasma gondii, Rickettsia, and the Helminths.

An antigen fused to a biotin-binding protein, e.g., a rhizavidin protein can be, e.g., a truncated pneumococcal PsaA protein, pneumolysin toxoid pneumococcal serine/threonine protein kinase (StkP), pneumococcal serine/threonine protein kinase repeating unit (StkPR), pneumococcal PcsB protein, staphylococcal alpha hemolysin, Mycobacterium tuberculosis mtb protein ESAT-6, M. tuberculosis cell wall core antigen, Chlamydia CT144, CT242 or CT812 polypeptides or fragments of these, Chlamydia DNA gyrase subunit B, Chlamydia sulfite synthesis/biphosphate phosphatase, Chlamydia cell division protein FtsY, Chlamydia methionyl-tRNA synthetase, Chlamydia DNA helicase (uvrD), Chlamydia ATP synthase subunit I (atpI), or Chlamydia metal dependent hydrolase.

An antigen fused to a biotin-binding protein, e.g., a rhazavidin protein can be from the pathogen Mycobacterium tuberculosis (TB), an intracellular bacterial parasite. One example of a TB antigen is TbH9 (also known as Mtb 39A). Other TB antigens include, but are not limited to, DPV (also known as Mtb8.4), 381, Mtb41, Mtb40, Mtb32A, Mtb64, Mtb83, Mtb9.9A, Mtb9.8, Mtb16, Mtb72f, Mtb59f, Mtb88f, Mtb71f, Mtb46f and Mtb31f, wherein “f” indicates that it is a fusion or two or more proteins.

An antigen can be derived from a Chlamydia species, e.g., Chlamydiaceae (consisting of Chlamydiae and Chlamydophila), are obligate intracellular gram-negative bacteria. Chlamydia trachomatis infections are among the most prevalent bacterial sexually transmitted infections, and perhaps 89 million new cases of genital chlamydial infection occur each year. The Chlamydia of the present invention include, for example, C. trachomatis, Chlamydophila pneumoniae, C. muridarum, C. suis, Chlamydophila abortus, Chlamydophila psittaci, Chlamydophila caviae, Chlamydophila felis, Chlamydophila pecorum, and C. pneumoniae. Animal models of chlamydial infection have established that T-cells play a critical role both in the clearance of the initial infection and in protection from re-infection of susceptible hosts. Hence, the immunogenic compositions as disclosed herein can be used to provide particular value by eliciting cellular immune responses against chlamydial infection.

More specifically, Chlamydial antigens include DNA gyrase subunit B, sulfite synthesis/biphosphate phosphatase, cell division protein FtsY, methionyl-tRNA synthetase, DNA helicase (uvrD); ATP synthase subunit I (atpI) or a metal-dependent hydrolase (U.S. Patent Application Pub. No. 20090028891). Additional Chlamydia trachomatis antigens include CT144 polypeptide, a peptide having amino acid residues 67-86 of CT144, a peptide having amino acid residues 77-96 of CT144, CT242 protein, a peptide having amino acids 109-117 of CT242, a peptide having amino acids 112-120 of CT242 polypeptide, CT812 protein (from the pmpD gene), a peptide having amino acid residues 103-111 of the CT812 protein; and several other antigenic peptides from C. trachomatis, as disclosed in WO 2009/020553 or US patent Application 2014/0154287, which are incorporated herein in their entirety by reference. See. Additionally, Chlamydia pneumoniae antigens including homologues of the foregoing polypeptides (see U.S. Pat. No. 6,919,187), can be used an antigens in the immunogenic compositions and methods as disclosed herein.

Fungal antigens can be derived from Candida species and other yeast; or other fungi (aspergillus, other environmental fungi). Regarding other parasites, malaria as well as worms and amoebae may provide the antigenic antigen for use in the in the immunogenic compositions and methods as disclosed herein.

An antigen is an anti-influenza immunogen, including a surface glycoprotein hemagglutinin (HA) or neuraminidase (NA). In some embodiments, an antigen can also include those used in biological warfare, such as ricin, which may provoke a CMI response.

Additionally, an antigen is an antigen expressed by a cancer or tumor, or derived from a tumor. In some embodiments, such antigens are referred to herein as a “cancer antigen” and are typically a protein expressed predominantly on the cancer cells, such that the conjugate elicits both potent humoral and potent cellular immunity to this protein. A large number of cancer-associated antigens have been identified, several of which are now being used to make experimental cancer treatment vaccines and are thus suitable for use in the present embodiments. Antigens associated with more than one type of cancer include Carcinoembryonic antigen (CEA); Cancer/testis antigens, such as NY-ESO-1; Mucin-1 (MUC1) such as Sialyl Tn (STn); Gangliosides, such as GM3 and GD2; p53 protein; and HER2/neu protein (also known as ERBB2). Antigens unique to a specific type of cancer include a mutant form of the epidermal growth factor receptor, called EGFRvIII; Melanocyte/melanoma differentiation antigens, such as tyrosinase, MART1, gp100, the lineage related cancer-testis group (MAGE) and tyrosinase-related antigens; Prostate-specific antigen; Leukaemia-associated antigens (LAAs), such as the fusion protein BCR-ABL, Wilms' tumour protein and proteinase 3; and Idiotype (Id) antibodies. See, e.g., Mitchell, 3 Curr. Opin. Investig. Drugs 150 (2002); Dao & Scheinberg, 21 Best Pract. Res. Clin. Haematol. 391 (2008).

In some embodiments, the methods and compositions and kits as disclosed herein are useful for isolating and purifying biotin-binding proteins, such as Rhizavidin proteins comprising SEQ ID NO: 1 or a protein of at least 80% sequence identity thereto, or fusion proteins thereof, that comprise a Staphylococcus aureus (SA) protein antigen. Such fusion proteins are disclosed in U.S. provisional application 62/477,618, filed on Marcg 28, 2017, and incorporated herein in its entirety by reference.

Another approach in generating an immune response against cancer employs antigens from microbes that cause or contribute to the development of cancer. These vaccines have been used against cancers including hepatocellular carcinoma (hepatitis B virus, hepatitis C virus, Opisthorchis viverrin), lymphoma and nasoparyngeal carcinoma (Epstei-Barr virus), colorectal cancer, stomach cancer (Helicobacter pylori), bladder cancer (Schisosoma hematobium), T-cell leukemia (human T-cell lymphtropic virus), cervical cancer (human papillomavirus), and others.

In some embodiments, an antigen is an antigen of autoimmune diseases, e.g., a “self-antigens.” Autoimmune diseases contemplated for diagnosis according to the assays described herein include, but are not limited to alopecia areata, ankylosing spondylitis, antiphospholipid syndrome, Addison's disease, aplastic anemia, multiple sclerosis, autoimmune disease of the adrenal gland, autoimmune hemolytic anemia, autoimmune hepatitis, autoimmune oophoritis and orchitis, Behcet's Disease, bullous pemphigoid, cardiomyopathy, celiac sprue-dermatitis, chronic fatigue syndrome, chronic inflammatory demyelinating syndrome (CFIDS), chronic inflammatory polyneuropathy, Churg-Strauss syndrome, cicatricial pemphigoid, CREST Syndrome, cold agglutinin disease, Crohn's disease, dermatitis herpetiformis, discoid lupus, essential mixed cryoglobulinemia, fibromyalgia, glomerulonephritis, Grave's disease, Guillain-Barre, Hashimoto's thyroiditis, idiopathic pulmonary fibrosis, idiopathic thrombocytopenia purpura (ITP), IgA nephropathy, insulin dependent diabetes (Type I), Lichen Planus, lupus, Meniere's Disease, mixed connective tissue disease, myasthenia gravis, myocarditis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff-man syndrome, Takayasu arteritis, temporal arteritis/giant cell arteritis, ulcerative colitis, uveitis, Wegener's syndrome, vasculitis and vitiligo. It is generally important to assess the potential or actual CMI responsiveness in subjects having, or suspected of having or being susceptible to an autoimmune disease.

In some embodiments, the methods and compositions and kits as disclosed herein can be used for possible selection of desirable cells using antibodies directed against the cells to be isolated, where the antibody is labeled with a biotin or biotin derivative, and where the biotin-binding domain, e.g., rhizavidin is bound to the biotin or biotin derivative. Accordingly, another aspect of the invention is that it opens up for many different post-isolation use of the cell-linked antibody/ligand. The released cells continue to carry their biotinylated antibodies.

One can envisage direct conjugation of antigenic proteins(i.e., an antigen) to the biotin-binding domains, e.g., a rhizavidin protein as disclosed herein instead of linking them by a peptide bond to form a fusion protein.

Following release of the binding pair, i.e. following release of the biotin-binding domain (e.g., rhizavidin protein), LA-matrix or support may be re-used.

In one embodiment, the solid phase comprises LA-containing magnetic particles and the magnetic particles and attached biotin-binding domain (e.g., rhizavidin protein) are isolated from a mixed or heterogeneous protein sample comprising the biotin-binding domain (e.g., rhizavidin protein) and other proteins by magnetic aggregation.

Purification procedures in which the method may be used include those conventionally used to separate cells, nucleic acids, proteins and other biomaterials, organic compounds, etc. The method may also be used for isolation followed by elution of biotin-binding domain (e.g., rhizavidin protein) complexes for further downstream analysis like Mass-spectroscopy. Applications in high throughput screening are also applicable.

All parameters involved in the attachment and release system described herein may vary dependent on biotin-binding domain (e.g., rhizavidin protein, fusion protein or complex thereof) to be isolated, the lipoic acid compound used, and also the type of solid phase used e.g. size of the magnetic beads. All conditions used may readily be determined by those skilled in the art for any given lipoic acid compound and any biotin-binding domain (e.g., rhizavidin protein, fusion protein or complex thereof) pairs used.

Conditions for the release or displacement of the biotin-binding domain (e.g., rhizavidin protein, fusion protein or complex thereof) from the lipoic acid compound may be varied as appropriate. The step of releasing the biotin-binding domain (e.g., rhizavidin protein, fusion protein or complex thereof) with a displacement ligand e.g. a free lipoic acid compound or alternative thereof (e.g., free biotin, or biotin-derivative or a fragment thereof) may take place in any convenient or desired way. The LA matrix or immobilized LA sample containing the bound biotin-binding domain (e.g., rhizavidin protein, fusion protein or complex thereof), conveniently in an aqueous medium, may simply be contacted with the displacement ligand, e.g. the displacement ligand may simply be added to a sample, and the reaction mixture allowed to stand under appropriate conditions for a time interval to allow the displacement ligand (i.e., free lipoic acid or biotin) to bind to the biotin-binding domain (e.g., rhizavidin protein, fusion protein or complex thereof), thereby separating the biotin-binding domain (e.g., rhizavidin protein, fusion protein or complex thereof) from the immobilized lipoic acid compound.

The amount of displacement ligand required for optimal release of the biotin-binding domain (e.g., rhizavidin protein, fusion protein or complex thereof) will of course vary depending upon the entities bound, their ratio, and the number or quantity of entities e.g. the amount of biotin-binding domain (e.g., rhizavidin protein, fusion protein or complex thereof) requiring isolation, and can readily be determined according to need.

For example, for purification and isolation of a rhizavidin protein, fusion protein or complex thereof as disclosed herein, the ratio of magnetic particles to amount of rhizavidin protein, fusion protein or complex thereof may vary in different systems and with different applications, and different amounts of the displacement ligand (i.e., lipoic acid compound) will accordingly be required to detach the rhizavidin protein, fusion protein or complex thereof from the particles. An example concentration of the lipoic acid displacement ligand could be 1 mg/ml, 5 mg/ml, or 10 mg/ml. Concentration of free lipoic acid, in the range of from about 1 to 10 mg/ml, preferably 2 to 5 mg/ml, has been found to be effective. Alternatively, in some embodiments, a concentration of free lipoic acid, in the range of from about lmg/ml, or about 1.5 mg/ml, or about 2.0 mg/ml, or about 2.5 mg/ml, or about 3 mg/ml or about 5 mg/ml or greater than 5 mg/ml has also been found to be effective.

Conditions for detachment using a free lipoic acid may also be varied as appropriate. Typically, incubation with a free lipoic acid compound will be effective at temperatures in the range from about 0° C. (i.e., on ice) to 37° C., or in some embodiments at room temperature (RT).

Incubation times for incubation of the LA-matrix (with bound rhizavidin protein, fusion protein or complex) with the displacement ligand (i.e., free lipoic acid) will vary depending on the temperature, materials, and concentrations used and may readily be determined by those skilled in the art for any given set of conditions. Short incubation times are attractive for the user. Typically, incubation times will range from about 2 to about 30 minutes, preferably from about 5 to 10 minutes for release of the rhizavidin protein, fusion protein or complex from the lipoic-acid immobilized to the solid support.

In some cases, it may be desirable to assist reversal of the linkage of the rhizavidin protein, fusion protein or complex linked to the immobilized lipoic acid, for example by gentle stirring or mixing e.g. pipetting in order to assist breakage of a linkage destabilized by the displacement ligand (i.e., free lipoic acid). Best results are obtained by incubating on an apparatus providing both gentle tilting and rotation.

The advantages of this more general method of reversing the binding of the rhizavidin protein, fusion protein or complex with the immobilized lipoic acid compound are self-evident and include the advantages of being more convenient, less time consuming and laborious to develop and therefore more cost-effective. Also the methods as disclosed herein are carried out under very mild/gentle conditions which neither lead to the destruction or loss of activity of the rhizavidin protein, fusion protein or complex thereof, nor affect the life or native status of any the rhizavidin protein, fusion protein or complex thereof.

### Kits

Another aspect of the present disclosure is directed towards kits. The kits can comprise a solid support, a lipoic acid compound and a displacement reagent (e.g., free lipoic acid). The solid support can generally be any solid support, as disclosed herein. For example, the solid support can be particulate, or magnetic particles. The solid support can further comprise at least one lipoic acid compound. The displacement reagent can generally be any material sufficient to displace the biotin-binding domain, e.g., a rhizavidin protein or fusion or complex comprising the same, from the LA which is immobilized to a solid support. For example, the displacement reagent can be a free lipoic acid compound, or other ligand which binds to the biotin-binding protein (e.g., a rhizavidin protein or fusion or complex comprising the same), such as biotin or a biotin derivative.

In some embodiments, the present disclosure relates to a kit comprising: (a) a lipoic acid compound attached to a solid support; and (b) at least one reagent (e.g., displacement ligand) to remove an immobilized rhizavidin protein or fusion protein comprising a rhizavidin protein from the lipoic acid compound attached to the solid support.

In some embodiments, the kit can further comprise an expression vector comprising the nucleic acid sequence encoding a Rhizavidin fusion protein, wherein the nucleic acid sequence comprises (i) a nucleic acid sequence encoding a rhizavidin protein comprising SEQ ID NO: 1 or a protein of at least 80% sequence identity to SEQ ID NO: 1, and (ii) a nucleic acid comprising a multiple insertion site (MIS) for insertion of a nucleic acid sequence encoding a protein of interest (e.g., an antigen) to be fused to the Rhizavidin protein. For example, the expression vector can comprise a nucleic acid comprising a multiple insertion site (MIS) that is located at the 5′- of the nucleic acid sequence encoding a rhizavidin protein comprising SEQ ID NO: 1 or a protein of at least 80% sequence identity to SEQ ID NO: 1 such that the protein of interest is at the N-terminus of the rhizavidin protein, therefore allowing generation of a rhizavidin-fusion protein wherein the fused protein is located at the N-terminus of the rhizavidin protein. In an alternative embodiment, the expression vector can comprise a nucleic acid comprising a multiple insertion site (MIS) that is located at the 3′- of the nucleic acid sequence encoding a rhizavidin protein comprising SEQ ID NO: 1 or a protein of at least 80% sequence identity to SEQ ID NO: 1, such that the protein of interest is at the C-terminus of the rhizavidin protein, therefore allowing generation of a rhizavidin-fusion protein wherein the fused protein is located at the C-terminus of the rhizavidin protein.

In some embodiments, the expression vector can optionally further comprise a nucleic acid sequence comprising a lipidation sequence at the 5′ or 3′ of the nucleic acid sequence encoding a rhizavidin protein comprising SEQ ID NO: 1 or a protein of at least 80% sequence identity to SEQ ID NO: 1. In some embodiments, a lipidation sequence increases the correct folding of a fusion protein and allows one to obtain a high yield of soluble recombinant protein. In some embodiments, a lipidation sequence comprises at least 10- or at least 15 or more than 15 amino acids of the following sequence: MKKVAAFVALSLLMAGC (SEQ ID NO: 6). In some embodiments, a lipidation sequence is located at the 5′ end of a nucleic acid sequence encoding a rhizavidin protein comprising SEQ ID NO: 1 or a protein of at least 80% sequence identity to SEQ ID NO: 1. In some embodiments, a lipidation can be added on the Cys residue of lipidation sequence by bacteria, e.g., E. coli, during the process of expression.

In some embodiments, the expression vector can optionally further comprise a nucleic acid sequence comprising a linker peptide between the nucleic acid sequence encoding a rhizavidin protein comprising SEQ ID NO: 1 or a protein of at least 80% sequence identity to SEQ ID NO: 1 and the nucleic acid comprising a multiple insertion site (MIS). In some embodiments, the expression vector can be used to generate a rhizavidin-fusion protein, comprising an antigenic peptide or antigen polypeptide.

The following examples are included to demonstrate preferred embodiments of the invention. It should be appreciated by those of skill in the art that the techniques disclosed in the examples which follow represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

Having generally described this invention, the same will become more readily understood by reference to the following specific examples which are included herein for purposes of illustration only and are not intended to be limiting unless otherwise specified.

### EXAMPLES

The examples presented herein relate to the methods of purification and isolation of a biotin-binding protein, e.g., rhizavidin using a lipoic acid affinity chromatography. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Abbreviations. The following abbreviations are used in the experiments:
PBS=Phosphate Buffered Saline
RT=Room temperature
Reagents & solutions:
Lipoic acid (LA) (Sigma, T1395-5G)
CARBOXYLINK™ Coupling Resin (Immobilized Diaminodipropylamine) (Life technologies, 20266); 4% crosslinked beaded agarose, supplied in a 50% slurry containing 0.02% sodium azide
EDC (Ethyl-3-[3-dimethylaminopropyl]carbodiimide hydrochloride) (Life technologies, 22980)
Sulfo-NHS (N-hydroxysulfosuccinimide) (Life technologies,24510)
Econo-Pac Chromatography Columns (Bio-Rad, 7321010EDU)
Solutions and Buffers:
Lipoic acid (LA) stock solution: 50 mg/ml Lipoic acid (Sigma, T1395-5G) in 100% ethanol.
0.1M MES buffer: 0.1M 2-(N-morpholino)ethanesulfonic acid (MES) at range of between 4.5-6, preferably at pH 5.0.
1M Na2HPO4: Na2HPO4 at 1M at pH 11 (pH adjusted with NaOH)
Coupling buffer: 50% ethanol+50% 0.1M MES buffer
1× PBS Buffer: 10 mM PO₄³⁻, 137 mM NaCl, and 2.7 mM KCl at pH7.5
Wash buffer: 50% ethanol, 20 mM Tris, 1M NaCl
Lysis buffer: 20 mM Tris, 1M NaCl, pH8.0
Elution buffer: 2.5 mg/ml of Lipoic acid (Sigma, T1395-5G) in 20 mM Tris, 1M NaCl, pH8, 5% ethanol.

### Example 1

Aspects of the present invention relate to technology to rapidly and efficiently purify a biotin-binding protein using affinity chromatography using a lipoic acid column. As an exemplary method, the inventors demonstrate highly efficient purification of a rhizavidin protein comprising SEQ ID NO: 1 from a column comprising lipoic acid. It is envisioned that other biotin-binding proteins, rhizavidin proteins, and rhizavidin complexes (e.g., rhizavidin-fusion proteins and or antigens cross-linked to rhizavidin) can be purified using the methods, composition and kits as disclosed herein.

In particular, the inventors discovered that the affinity of rhizavidin for lipoic acid is approximately the same as histadine, e.g., a polyhistadine tag, (also referred to as a hexa histidine-tag, 6× His-tag, His6 tag or HIS-TAG™) for nickel. A polyhidtadine-tag can bind to a sepharose/agarose functionalized with a chelator, such as iminodiacetic acid (Ni-IDA) and nitrilotriacetic acid (Ni-NTA) with micromolar affinity. While a protein with His-tag can be eluted using pH titration, as His-tag binds to the nickel at a high pH, but at a pH of ˜4, the histadine becomes protonated and is completed off the metal ion. Thus, purification with a His-tag requires a low and acidic pH and can lead to protein denaturing or incorrect folding of the protein to be purified.

Herein, the inventors have discovered that lipoic acid can be used to bind to rhizavidin with a micromolar affinity, and can be removed by competitive inhibition using a lipoic acid of between 1.0 mg/ml and 10.0 mg/ml in an elution buffer, at a pH of between about 7.5 and 9.0, therefore achieving a similar purification efficiency as a Ni-NTA column purification using a His tag, but without the need for low or acidic pH for elution, therefore minimizing risk of protein denaturation.

Additionally, the inventors assessed the binding of rhizavidin to several ligands for use in affinity chromatography purification. Surprisingly, while rhizavidin is known to bind to biotin and other biotin-related or biotin-derivatives, the inventors discovered that rhizavidin did not bind to a number of biotin-derivatives, such as, for example, the biotin-derivatives HABA (hydrooxyazobenzene-benzoic acid) or dimethyl-HABA. Moreover, the inventors discovered that columns that comprising HABA or dimethyl-HABA were not effective at purifying rhizavidin (data not shown). Accordingly, only lipoic acid was discovered to be effective for efficient purification of a rhizavidin protein or fusion protein.

Preparation of the Lipoic Acid Resin (IA-Matrix)

The following steps can be followed to prepare a LA-matrix for use in the methods as disclosed herein:
i. Resuspend CARBOXYLINK™ Coupling Resin (Life technologies, 20266) and aliquot 1 ml of suspended resin into an empty column, e.g., a bio-rad Econo-Pac Chromatography Column, allow resin to settle. Wash the resin with approx.20 ml of water and add 10 ml of PBS (×1) to equilibrate resin.
ii. Prepare a 100 mg/ml solution of Sulfo-NHS by dissolving 50 mg of Sulfo-NHS (N-hydroxysulfosuccinimide) in 0.5 ml of 0.1M MES buffer.
iii. Prepare a Sulfo-NHS/LA mixture by adding 0.25 ml of ethanol and 0.25 ml of 50 mg/ml LA stock solution (12.5 mg of LA) to 0.5 ml of Sulfo-NHS (100 mg/ml).
iv. To cross link the LA to the resin, dissolve 25 mg of EDC in 0.75 ml of coupling buffer and immediately add to Sulfo-NHS/LA mixture. Incubate the EDC/Sulfo-NHS/LA mixture with rotation for 30 min RT. Adjust the pH of the EDC/Sulfo-NHS/LA mixture to pH 7.0 by adding 1M Na2HPO4 (pH11) (approx. 0.5 ml of 1M Na2HPO4)
v. Resuspend the equilibrated CARBOXYLINK™ Coupling Resin with EDC/Sulfo-NHS/LA mixture and transfer everything to a 5 ml tube, incubate with rotation overnight RT.
vi. Transfer resin into an empty column, allow LA resin/LA matrix to settle. Wash resin with the following reagents in the following order; (i) 20 ml of wash buffer, (ii) 20 ml of H2O, (iii) Wash resin comprising 5 ml of 20% ethanol. Store LA resin/LA matrix in 20% ethanol until use.

Purification of Rhizavidin or Rhizavidin Fusion Protein by IA-Matrix

The following steps can be followed in the methods as disclosed herein to purify a biotin-binding protein, e.g., rhizavidin or a rhiavidin fusion protein using a LA-matrix from a bacterial suspension expressing the rhizavidin or a rhiavidin fusion protein:
v. Collect E. coli culture by centrifugation, discard supernatant, and resuspend pelleted cells with a lysis buffer (20 mM Tris, 1M NaCl, pH8.0) containing DNase, proteinase inhibitors and 10 mM MgCl₂. Lyse bacterial cells by sonication or other approach and collect cell lysate by centrifugation.
vi. Pack 0.5 ml of LA resin into an empty column and wash the LA resin with 20 ml of H₂O. Equilibrate resin with 20 ml of lysis buffer (20 mM Tris, 1M NaCl, pH8.0).
vii. Add cell lysate from step (i) to the LA resin and mix well and incubate for >2 h at 4° C., while shaking.
viii. Transfer resin into an empty column after incubation and wash the LA resin with wash buffer (50% ethanol, 20 mM Tris, 1M NaCl) until no protein is detected in flow through
ix. Elute protein with elution buffer (2.5 mg/ml of Lipoic acid in 20 mM Tris, 1M NaCl, 5% ethanol at pH8).

1. A method of reversibly immobilizing a rhizavidin protein or a fusion protein comprising a rhizavidin protein to a solid support, comprising contacting the support with the rhizavidin protein or a fusion protein comprising a rhizavidin protein, wherein the solid support comprises a lipoic acid (LA) compound on the surface of the solid support.

2. The method of claim 1, wherein the rhizavidin protein or a fusion protein comprising a rhizavidin protein is contacted with, or binds to the solid support in a solution having a pH between 5.5-9.0, or a solution comprising 1M NaCl.

3. The method of claim 1, further comprising contacting the solid support comprising the immobilized rhizavidin protein or rhizavidin protein fusion protein with an elution buffer comprising 1-10 mg/ml of a lipoic acid (LA) compound to release the rhizavidin protein or fusion protein thereof from the solid support.

4. The method of claim 1, comprising the steps, in the order of:
(i) contacting the solid support that comprises a lipoic acid (LA) compound on the surface of the solid the support with a solution comprising the rhizavidin protein or the fusion protein comprising a rhizavidin protein;
(ii) incubating for a sufficient amount of time to allow the rhizavidin protein or the fusion protein comprising a rhizavidin protein to bind to the lipoic acid (LA) compound;
(iii) washing the solid support comprising the lipoic acid (LA) compound on the surface of the solid the support with a wash solution to remove non-bound rhizavidin protein or a fusion protein comprising a rhizavidin protein;
(iv) contacting the solid support comprising the immobilized rhizavidin protein or rhizavidin protein fusion protein with an elution buffer comprising 1-10 mg/ml of a lipoic acid (LA) and separating portions of the elution buffer which comprises the rhizavidin protein or fusion protein released from the solid support from portions of the elution buffer which do not comprise the rhizavidin protein or fusion protein released from the solid support; and
(v) collecting the portion of the elution buffer which comprises the rhizavidin protein or fusion protein thereof.

5. The method of claim 4, wherein the solution comprising a rhizavidin protein or a fusion protein comprising a rhizavidin protein that contacts the solid support comprising the lipoic acid (LA) compound on the surface of the solid the support in step (i) has a pH between 5.5-9.0, or a solution comprising 1M NaCl.

6. The method of claim 4, wherein the elution buffer comprises between 1.0-10 mg/ml of a lipoic acid (LA) compound to release the rhizavidin protein or fusion protein thereof from the solid support.

7. The method of claim 1, wherein the Rhizavidin protein comprises amino acids of SEQ ID NO: 1 or protein of at least 80% sequence identity to SEQ ID NO: 1.

8. The method of claim 1, wherein the solid support is selected from the group consisting of: plastic, glass, ceramics, silicone, metal, cellulose, membranes, gels, a particle, a magnetic particle or a SEPHAROSE™ bead.

9. (canceled)

10. (canceled)

11. The method of claim 1, wherein the lipoic acid compound is directly bound or linked to the solid support via a covalent bond, or indirectly linked to the solid support via a protein linker, peptide, nucleic acid, oligosaccharideoligosachharide, glycoprotein, or cross-linking reagent.

12. The method of claim 1, wherein the lipoic acid compound is selected from any of:
(i) lipoic acid,
(ii) alpha-lipoic acid (ALA),
(iii) a lipoic acid derivative,
(iv) a racemic lipoic acid, or enantiomerically pure or enantiomerically enriched R (+)-alpha-lipoic acid or S-(−)-alpha-lipoic acid, and
(iv) lipoic acid derivative is selected from the group of: Lipoylpyridoxamine, Lipoylpyridoxamine hydrochloride, Lipoylpyridoxamine hydrobromide, Lipoylpyridoxamine methanesulfonate, Lipoylpyridoxamine p-toluenesulfonate, 1,2-dithiolane analog, diethoxycarbonylated lipoic acid, 6,8-Bisacetylmercaptooctanoic Acid (Bis-acetyl Lipoic Acid), 6,8-Bisbenzoylmercaptooctanoic Acid (Bisbenzoyl Lipoic acid), 8-Acetylmercapto-6-mercaptooctanoic Acid (Monoacetyl Lipoate), 6,8-Biscarbamoylmethylmercaptooctanoic Acid, 6,8-Bis-[S—(N-methylsuccinimido)]mercaptooctanoic Acid.

13.-15. (canceled)

16. The method of claim 1, further comprising eluting the rhizavidin protein or rhizavidin protein fusion protein from the solid support by contacting the solid support comprising the immobilized rhizavidin protein or rhizavidin protein fusion protein with an elution buffer comprising 1-10 mg/ml of a lipoic acid (LA) compound to release the rhizavidin protein or fusion protein thereof from the solid support.

17. A kit comprising:
a. a lipoic acid compound attached to a solid support; and
b. at least one reagent to remove an immobilized rhizavidin protein or fusion protein comprising a rhizavidin protein from the lipoic acid compound attached to the solid support.

18. The kit of claim 17, further comprising an expression vector comprising the nucleic acid sequence for expression of a Rhizavidin fusion protein, wherein the nucleic acid sequence comprises (i) a nucleic acid sequence encoding a rhizavidin protein comprising SEQ ID NO: 1 or a protein of at least 80% sequence identity to SEQ ID NO: 1, and (ii) a nucleic acid comprising a multiple insertion site (MIS) for insertion of a nucleic acid sequence encoding a protein of interest to be fused to the Rhizavidin protein.

19. (canceled)

20. (canceled)

21. The kit of claim 18, wherein the expression vector comprises any one or more of:
(i) a multiple insertion site (MIS) that is at the 5′- of the nucleic acid sequence encoding a rhizavidin protein comprising SEQ ID NO: 1 or a protein of at least 80% sequence identity to SEQ ID NO: 1 such that the protein of interest is at the N-terminus of the rhizavidin protein,
(ii) a multiple insertion site (MIS) that is at the 3′- of the nucleic acid sequence encoding a rhizavidin protein comprising SEQ ID NO: 1 or a protein of at least 80% sequence identity to SEQ ID NO: 1 such that the protein of interest is at the C-terminus of the rhizavidin protein,
(iii) a nucleic acid sequence comprising a lipidation sequence at the 5′ of the nucleic acid sequence encoding a rhizavidin protein comprising SEQ ID NO: 1 or a protein of at least 80% sequence identity to SEQ ID NO: 1 and
(iv) a nucleic acid sequence comprising a linker peptide between the nucleic acid sequence encoding a rhizavidin protein comprising SEQ ID NO: 1 or a protein of at least 80% sequence identity to SEQ ID NO:
1 and the nucleic acid comprising a multiple insertion site (MIS).

22. (canceled)

23. The kit of claim 18, wherein the protein of interest is an antigenic peptide or antigen polypeptide.

24. The kit of claim 17, wherein solid support is selected from the group consisting of: plastic, glass, ceramics, silicone, metal, cellulose, membranes gels, a particle or a magnetic particle or a SEPHAROSE™ bead.

25. (canceled)

26. (canceled)

27. A composition comprising a solid support, a lipoic acid compound and a Rhizavidin protein or Rhizavidin fusion protein, wherein the lipoic acid compound is attached to the solid support, and the Rhizavidin protein or Rhizavidin fusion protein comprises at least amino acids of SEQ ID NO: 1 or protein of at least 80% sequence identity to SEQ ID NO: 1 and is bound to the lipoic acid compound.

28. The composition of claim 27, wherein the lipoic acid compound is selected from any of the group of:
(i) lipoic acid,
(ii) alpha-lipoic acid (ALA),
(iii) a lipoic acid derivative,
(iv) a racemic lipoic acid, or enantiomerically pure or enantiomerically enriched R (+)-alpha-lipoic acid or S-(−)-alpha-lipoic acid, and
(iv) lipoic acid derivative is selected from the group of: Lipoylpyridoxamine, Lipoylpyridoxamine hydrochloride, Lipoylpyridoxamine hydrobromide, Lipoylpyridoxamine methanesulfonate, Lipoylpyridoxamine p-toluenesulfonate, 1,2-dithiolane analog, diethoxycarbonylated lipoic acid, 6,8-Bisacetylmercaptooctanoic Acid (Bis-acetyl Lipoic Acid), 6,8-Bisbenzoylmercaptooctanoic Acid (Bisbenzoyl Lipoic acid), 8-Acetylmercapto-6-mercaptooctanoic Acid (Monoacetyl Lipoate), 6,8-Biscarbamoylmethylmercaptooctanoic Acid, 6,8-Bis-[S—(N-methylsuccinimido)]mercaptooctanoic Acid.

29.-31. (canceled)

32. The composition of claim 27, wherein the lipoic acid compound is attached to the solid support via a direct linkage of a covalent bond, or is indirectly linked to the solid support via a protein linker, peptide, nucleic acid, oligosaccharide, glycoprotein, or cross-linking reagent.

33. The composition of claim 27, wherein solid support is selected from any of:
(i) comprises any one of: plastic, glass, ceramics, silicone, metal, cellulose, membranes, gels, a particle or a magnetic particle,
(ii) in the form of any of: particles, sheets, dip-sticks, gels, filters, membranes, microfibre strips, biochips, tubes, wells, plates, fibre or capillaries, comb, pipette tip, microarrays, and
(iii) is a polymeric material selected from the group of: agarose, SEPHAROSE™, cellulose, nitrocellulose, alginate, Teflon, latex, acrylamide, nylon membranes, plastic, polystyrene, glass or silica or metals, and
(iv) is a SEPHAROSE™ bead.

34.-38. (canceled)

39. The composition of claim 27, further comprising one or more of:
(i) a buffer solution having a pH between 5.5-9.0 or
(ii) a solution comprising 1M NaCl.

40. (canceled)

41. The composition of claim 27 configured as an affinity chromatography column, wherein the column comprises the solid support with the attached, lipoic acid compound, and the Rhizavidin protein or Rhizavidin fusion protein bound to the lipoic acid compound.

42.-47. (canceled)


SEQUENCE LISTING (26 sequences)

SEQ ID NO: 1 - protein, 135 aa, Rhizobium etli
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 2 - protein, 20 aa, Rhizobium etli
MIRTNAVAALVFAVATSALA

SEQ ID NO: 3 - skipped

SEQ ID NO: 4 - protein, 176 aa, Rhizobium etli
MIITSLYATFGTIADGRRTSGGKTMIRTNAVAALVFAVATSALAFDASNFKDFSSIASASSSWQNQSGSTMIIQVDSFGNVSGQYVNRAQGTGCQNSPYPLTGRVNGTFIAFSVGWNSTENCNSATGWTGYAQVNGNTEIVTSWLAYEGGSGPAIEQGQDTFQYVPTTENKSLLKD

SEQ ID NO: 5 - protein, 44 aa, Rhizobium etli
MIITSLYATFGTIADGRRTSGGKTMIRTNAVAALVFAVATSALA

SEQ ID NO: 6 - protein, 17 aa, Artificial Sequence
  Description of Artificial Sequence: Synthetic peptide
MKKVAAFVALSLLMAGC

SEQ ID NO: 7 - protein, 19 aa, Escherichia coli
MKKIWLALAGLVLAFSASA

SEQ ID NO: 8 - protein, 23 aa, Escherichia coli
MKKIWLALAGLVLAFSASAAQDP

SEQ ID NO: 9 - protein, 17 aa, Artificial Sequence
  Description of Artificial Sequence: Synthetic peptide
MKKVAAFVALSLLMAGC

SEQ ID NO: 10 - protein, 19 aa, Artificial Sequence
  Description of Artificial Sequence: Synthetic peptide
MKKIWLALAGLVLAFSASA

SEQ ID NO: 11 - protein, 23 aa, Artificial Sequence
  Description of Artificial Sequence: Synthetic peptide
MAPFEPLASGILLLLWLIAPSRA

SEQ ID NO: 12 - protein, 4 aa, Artificial Sequence
  Description of Artificial Sequence: Synthetic peptide
AQDP

SEQ ID NO: 13 - protein, 4 aa, Artificial Sequence
  Description of Artificial Sequence: Synthetic peptide
VSDP

SEQ ID NO: 14 - protein, 6 aa, Artificial Sequence
  Description of Artificial Sequence: Synthetic 6xHis tag
HHHHHH

SEQ ID NO: 15 - protein, 22 aa, Artificial Sequence
  Description of Artificial Sequence: Synthetic peptide
GGGGSSSVDKLAAALEHHHHHH

SEQ ID NO: 16 - protein, 9 aa, Artificial Sequence
  Description of Artificial Sequence: Synthetic peptide
VDKLAAALE

SEQ ID NO: 17 - protein, 16 aa, Artificial Sequence
  Description of Artificial Sequence: Synthetic peptide
GGGGSSSVDKLAAALE

SEQ ID NO: 18 - protein, 14 aa, Artificial Sequence
  Description of Artificial Sequence: Synthetic peptide
VDKLAAALEHHHHH

SEQ ID NO: 19 - protein, 22 aa, Artificial Sequence
  Description of Artificial Sequence: Synthetic peptide
GGGGSSSVDKLAAALEHHHHHH

SEQ ID NO: 20 - protein, 14 aa, Artificial Sequence
  Description of Artificial Sequence: Synthetic peptide
GSPGISGGGGGILE

SEQ ID NO: 21 - protein, 21 aa, Artificial Sequence
  Description of Artificial Sequence: Synthetic peptide
MNSKKLCCICVLFSLLAGCAS

SEQ ID NO: 22 - protein, 19 aa, Artificial Sequence
  Description of Artificial Sequence: Synthetic peptide
MRYSKLTMLIPCALLLSAC

SEQ ID NO: 23 - protein, 24 aa, Artificial Sequence
  Description of Artificial Sequence: Synthetic peptide
MFVTSKKMTAAVLAITLAMSLSAC

SEQ ID NO: 24 - protein, 18 aa, Artificial Sequence
  Description of Artificial Sequence: Synthetic peptide
MIKRVLVVSMVGLSLVGC

SEQ ID NO: 25 - protein, 4 aa, Artificial Sequence
  Description of Artificial Sequence: Synthetic peptide
VSDP

SEQ ID NO: 26 - protein, 4 aa, Artificial Sequence
  Description of Artificial Sequence: Synthetic peptide
AQDP